(19) United States
(12) Patent Application Publication
(10) Pub. No.: US 2021/0315796 A1
(43) Pub. Date: Oct. 14, 2021

(54) BIPHASIC TOPICAL COMPOSITIONS AND METHODS OF USE

(71) Applicant: RODAN & FIELDS, LLC, San Francisco, CA (US)

(72) Inventors: Christine Marie CRANE, Walnut Creek, CA (US); Olga DUEVA-KOGANOV, White Plains, NY (US); Taylor OSWALD, Oakland, CA (US); Ying Min WU, Oakland, CA (US)

(21) Appl. No.: 17/223,140

(22) Filed: Apr. 6, 2021

Related U.S. Application Data
(60) Provisional application No. 63/008,209, filed on Apr. 10, 2020.

(51) Int. Cl. A61K 8/92; A61K 8/34; A61Q 19/02; A61K 8/49; A61K 8/37; A61K 8/42; A61K 8/67; A61Q 19/04; A61K 9/00; A61K 47/10; A61K 47/44

(52) U.S. Cl. CPC A61K 8/922; A61K 2800/5922; A61K 8/345; A61Q 19/02; A61K 8/498; A61K 8/37; A61K 8/4926; A61K 8/42; A61K 8/678; A61K 8/676; A61Q 19/04; A61K 9/0014; A61K 47/10; A61K 47/44; A61K 2800/31; A61K 8/34

(57) ABSTRACT

The biphasic topical compositions of various embodiments comprise a Phase A including castor oil and one or more additional non-polar solvents, and a Phase B including ethanol and one or more additional polar solvents, wherein the ratio of castor oil to ethanol is about 1:1 to about 3:1; and wherein the biphasic topical composition is free of surfactants and emulsifiers. The methods include treating uneven skin tone, treating acne, lightening or brightening the skin, protecting a subject's skin from sun damage, protecting a subject's skin and scalp from the effects of aging, improving the look of the skin, improving the appearance of the skin, treating fibrotic skin conditions, treating the hair, and improving the appearance of the hair.

### PRIORITY CLAIM

This application claims the benefit of priority of U.S. Provisional Application 63/008,209, filed Apr. 10, 2020, the contents of which are hereby incorporated by reference

### SUMMARY

Embodiments disclosed herein are directed to biphasic topical composition comprising: a Phase A including castor oil and one or more additional non-polar solvents, and a Phase B including ethanol and one or more additional polar solvents, wherein the ratio of castor oil to ethanol is about 1:1 to about 3:1; and wherein the biphasic topical composition is free of surfactants and emulsifiers. In certain embodiments, the biphasic topical composition may further comprise one or more active ingredients.

Embodiments disclosed herein are directed to methods of treating uneven skin tone in a subject in need thereof comprising: shaking a biphasic topical composition comprising a Phase A including castor oil, one or more additional non-polar solvents, and one or more oil soluble active ingredients, and a Phase B including ethanol, one or more additional polar solvents, and one or more polar or water soluble active ingredients to form a mixed biphasic topical composition, applying the mixed biphasic topical composition to the skin of the subject, wherein the ratio of castor oil to ethanol in the biphasic topical composition is about 1:1 to about 3:1. In certain embodiments, the biphasic topical composition and the mixed biphasic topical composition may further comprise one or more active ingredients.

Embodiments disclosed herein are directed to methods of lightening or brightening the skin in a subject in need thereof comprising: shaking a biphasic topical composition comprising a Phase A including castor oil, one or more additional non-polar solvents, and one or more oil soluble active ingredients, and a Phase B including ethanol, one or more additional polar solvents, and one or more polar or water soluble active ingredients to form a mixed biphasic topical composition, applying the mixed biphasic topical composition to the skin of the subject, wherein the ratio of castor oil to ethanol in the biphasic topical composition is about 1:1 to about 3:1. In certain embodiments, the biphasic topical composition and the mixed biphasic topical composition may further comprise one or more active ingredients.

Embodiments disclosed herein are directed to methods of protecting a subject's skin from sun damage comprising: shaking a biphasic topical composition comprising a Phase A including castor oil, one or more additional non-polar solvents, and one or more oil soluble active ingredients, and a Phase B including ethanol, one or more additional polar solvents, and one or more polar or water soluble active ingredients to form a mixed biphasic topical composition, applying the mixed biphasic topical composition to the skin of the subject, wherein the ratio of castor oil to ethanol in the biphasic topical composition is about 1:1 to about 3:1. In certain embodiments, the biphasic topical composition and the mixed biphasic topical composition may further comprise one or more active ingredients.

Embodiments disclosed herein are directed to methods of protecting a subject's skin from the effects of aging comprising: shaking a biphasic topical composition comprising a Phase A including castor oil, one or more additional non-polar solvents, and one or more oil soluble active ingredients, and a Phase B including ethanol, one or more additional polar solvents, and one or more polar or water soluble active ingredients to form a mixed biphasic topical composition, applying the mixed biphasic topical composition to the skin of the subject, wherein the ratio of castor oil to ethanol in the biphasic topical composition is about 1:1 to about 3:1. In certain embodiments, the biphasic topical composition and the mixed biphasic topical composition may further comprise one or more active ingredients.

### BRIEF DESCRIPTION OF THE DRAWINGS

For a fuller understanding of the nature and advantages of the present embodiments, reference should be made to the following detailed description taken in connection with the accompanying drawings, in which:

FIG. 1a is a graphical representation of trends in the separation time of niacinamide containing biphasic compositions (Examples 1-7) as a function of the ratio of Phase A to Phase B. FIG. 1b is a graphical representation of trends in the separation time of niacinamide containing biphasic compositions (Examples 1-7) as a function of the ratio of Castor Oil to Ethanol. FIG. 1c is a graphical representation of trends in the separation time of niacinamide containing biphasic compositions (Examples 1-7) as a function of the % Phase B.

FIG. 2a is a graphical representation of trends in the separation time of ascorbic acid containing biphasic compositions (Examples 8-11) as a function of the ratio of Phase A to Phase B. FIG. 2b is a graphical representation of trends in the separation time of ascorbic acid containing biphasic compositions (Examples 8-11) as a function of the ratio of Castor Oil to Ethanol. FIG. 2c is a graphical representation of trends in the separation time of ascorbic acid containing biphasic compositions (Examples 8-11) as a function of the % Phase B.

FIG. 3a is a graphical representation of trends in the separation time of ascorbic acid containing biphasic compositions (Examples 12-15) as a function of the ratio of Phase A to Phase B. FIG. 3b is a graphical representation of trends in the separation time of ascorbic acid containing biphasic compositions (Examples 12-15) as a function of the ratio of Castor Oil to Ethanol. FIG. 3c is a graphical representation of trends in the separation time of ascorbic acid containing biphasic compositions (Examples 12-15) as a function of the % Phase B.

### DETAILED DESCRIPTION

The state of the art has focused on developing topical compositions in the form of oil-in-water and water-in-oil emulsions where both water-soluble and oil-soluble ingredients are to be included in a single, pre-mixed (or a single-phase) composition. However, such compositions may not remain stable and may separate over time. Additionally, there exists many incompatible skin care ingredients that would be beneficial to apply simultaneously and be sold in a single container. Further, consumers are interested in products that are beneficial for sensitive skin and are made with natural ingredients. Developing topical compositions that could be provided to the consumer in a form that would provide long-term stability to the components contained therein and ease of use to the consumer would be an improvement to the topical compositions currently available.

The present disclosure is directed toward a biphasic topical composition comprising an oily phase, a combination of non-polar solvents referred to as Phase A, with dissolved oil soluble ingredients, and a polyol or water phase, a combination of polar solvents referred to as Phase B, with dissolved polyol or water-soluble ingredients. The biphasic topical composition is imparted with an improved transient miscibility due to the addition of an optimized ratio of a polar oil in Phase A and an alcohol in Phase B. This optimized ratio of polar oil and alcohol allows for the topical composition, once shaken, to form an emulsion long enough for said formulation to be applied on keratinous fibers evenly. In the absence of this combination of Phase A and Phase B of the composition separates too quickly to be applied evenly on the skin after shaking, which is an undesirable side effect as application of the product is uneven. Additionally, the topical compositions described herein lack emulsifiers and thickeners lowering overall costs for the final composition as well as processing costs and viscosity.

Physical separation of ingredients based on solubility allows for the combination of ingredients, that are incompatible together in a single formula, to be supplied in a container having two phases. Further, using a biphasic topical composition provides a fluid (low viscosity) composition that is easy to apply on the skin which is a desirable consumer attribute from an aesthetics point of view.

Oil-soluble and water-soluble ingredients of interest are those known to have a cosmetic or pharmaceutical benefit. Examples of ingredients beneficial in the system include combinations of niacinamide in the water or polyol phase with hydrophobically modified ascorbic acid in the oil phase. Additionally, skin benefits are seen using L-ascorbic acid in the water or polyol phase and phenethyl resorcinol in the oil phase for treating uneven skin tone or salicylic acid in the oil phase for treating acne. The biphasic topical compositions described herein also place potentially oxidatively unstable ingredients in the bottom, Phase B, layer away from the dispenser and air, enhancing long term shelf life of the composition.

The biphasic topical compositions embodiments described herein allow for the use of up to 40% glycerin without feeling tacky or sticky on the skin. This is imparted because the glycerin phase is the internal phase upon mixing, the addition of castor oil increases the skin absorption of the formulation, and the ethanol flashes off the skin at body temperature.

Various aspects will be described in detail hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 wt % to 8 wt % is stated, it is intended that 2 wt %, 3 wt %, 4 wt %, 5 wt %, 6 wt %, and 7 wt % are also explicitly disclosed, as well as the range of values greater than or equal to 1 wt % and the range of values less than or equal to 8 wt %.

All percentages, parts and ratios are based upon the total weight of the formulations and compositions and all measurements made are at about 25° C., unless otherwise specified.

The singular forms “a,” “an,” and “the” include plural referents unless the context clearly dictates otherwise. Thus, for example, reference to a “polymer” includes a single polymer as well as two or more of the same or different polymers; reference to an “excipient” includes a single excipient as well as two or more of the same or different excipients, and the like.

The word “about” when immediately preceding a numerical value means a range of plus or minus 10% of that value, e.g, “about 50” means 45 to 55, “about 25,000” means 22,500 to 27,500, etc, unless the context of the disclosure indicates otherwise, or is inconsistent with such an interpretation. For example, in a list of numerical values such as “about 49, about 50, about 55, “about 50” means a range extending to less than half the interval(s) between the preceding and subsequent values, e.g, more than 49.5 to less than 52.5. Furthermore, the phrases “less than about” a value or “greater than about” a value should be understood in view of the definition of the term “about” provided herein.

The phrase “active ingredient” refers to any bio-functional agent which provides a benefit to the subject.

The terms “administer,” “administering” and “administration” as used herein refer to either directly administering a compound (also referred to as an agent of interest) or pharmaceutically acceptable salt of the compound (agent of interest) or a composition to a subject.

The term “excipients” as used herein encompasses carriers and diluents, meaning a material, composition or vehicle, such as a liquid or solid filler, diluent, excipient, solvent or encapsulating material involved in carrying or transporting a pharmaceutical, cosmetic or other agent across a tissue layer such as the stratum corneum or stratum spinosum.

The transitional term “comprising,” which is synonymous with “including,” “containing,” or “characterized by,” is inclusive or open-ended and does not exclude additional, un-recited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed subject matter. In some embodiments or claims where the term comprising is used as the transition phrase, such embodiments can also be envisioned with replacement of the term “comprising” with the terms “consisting of” or “consisting essentially of.”

The term “cosmetic” means an agent utilized, and/or intended to be applied to the human body for cleansing, beautifying, promoting attractiveness, altering the appearance of the skin or any combination thereof.

The term “composition” as used herein refers to a combination or a mixture of two or more different ingredients, components, or substances.

The term “keratinous fiber” as used herein refers to any tissue which contain keratin as a fibrous structural protein, including, but not limited to, skin, hair, and nails.

As used herein, the phase “oil soluble” refers to ingredients or compounds that are soluble in oil and also ingredients and compounds that are oil-dispersible.

The phrase “pharmaceutically acceptable” or “cosmetically acceptable” is employed herein to refer to those agents of interest/compounds, salts, compositions, dosage forms, etc, which are within the scope of sound medical judgment suitable for use in contact with the tissues of human beings and/or other mammals without excessive toxicity, irritation, allergic response, or other problem or complication, commensurate with a reasonable benefit/risk ratio. In some aspects, pharmaceutically acceptable means approved by a regulatory agency of the federal or a state government, or listed in the U.S. Pharmacopeia or other generally recognized pharmacopeia for use in mammals (e.g, animals), and more particularly, in humans.

The terms “patient” and “subject” are interchangeable and may be taken to mean any living organism which may be administered and/or treated with compounds or compositions provided for herein. As such, the terms “patient” and “subject” may comprise, but is not limited to, any non-human mammal, primate or human. In some embodiments, the “patient” or “subject” is a mammal, such as mice, rats, other rodents, rabbits, dogs, cats, swine, cattle, sheep, horses, primates, or humans. In some embodiments, the patient or subject is an adult, child, or infant. In some embodiments, the patient or subject is a human.

The term “substantially free” as used herein refers to the complete or near complete lack of an ingredient or component. For example, a composition that is “substantially free” of water would either completely lack water, or so nearly completely lack water that the effect would be the same as if it completely lacked water. In other words, a composition that is “substantially free” of an ingredient may still actually contain such a component as long as there is no measurable effect thereof.

The terms “topically” and “topical” as used herein refer to application of the compositions to the surface of the skin, mucosal cells, keratins and tissues. Examples of keratins are nails and hair.

The term “treating” as used herein, refers to methods of treating a skin disorder or a systemic condition, and generally includes the administration of a compound or composition which reduces the frequency of, or delays the onset of, symptoms of a medical condition or enhance the texture, appearance, color, sensation, or hydration of the intended tissue treatment area of the tissue surface in a subject relative to a subject not receiving the compound or composition. This can include reversing, reducing, or arresting the symptoms, clinical signs, and underlying pathology of a condition in a manner to improve or stabilize a subject's condition.

As used herein, the phase “water soluble” refers to ingredients or compounds that are soluble in water and also ingredients and compounds that are water-dispersible.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

### Compositions

Embodiments described herein are directed to a biphasic topical composition comprising: a Phase A including castor oil and one or more additional non-polar solvents, and a Phase B including ethanol and one or more additional polar solvents, wherein the ratio of castor oil to ethanol is about 1:1 to about 3:1; and wherein the biphasic topical composition is free of surfactants and emulsifiers.

In embodiments described herein, the ratio of Phase A to Phase B is selected from the group consisting of about 1:0.8 to about 1:3, about 1:1 to about 1:2.8, about 1:1.1 to about 1:2.6, about 1:1.2 to about 1:2.4, about 1:1.3 to about 1:2.2, about 1:1.4 to about 1:2, about 1:1.5 to about 1:1.8, and about 1:1.6 to about 1:1.7. In embodiments described herein, the ratio of Phase A to Phase B is about 1:1.1 to about 1:1.65. In embodiments described herein, the ratio of Phase A to Phase B is about 1:1.1. In embodiments described herein, the ratio of Phase A to Phase B is about 1:1.27. In embodiments described herein, the ratio of Phase A to Phase B is about 1:1.37. In embodiments described herein, the ratio of Phase A to Phase B is about 1:1.49. In embodiments described herein, the ratio of Phase A to Phase B is about 1:1.61. In embodiments described herein, the ratio of Phase A to Phase B is about 1:1.64. In embodiments described herein, the ratio of Phase A to Phase B is about 1:1.65. In embodiments described herein, the ratio of Phase A to Phase B is about 1:1.8.

In embodiments described herein, the ratio of oil to polyol is selected from the group consisting of about 1.2:1 to about 1:2.0, about 1.1:1 to about 1:1.9, about 1:1 to about 1:1.8, about 1:1 to about 1:1.7, about 1:1 to about 1:1.6, about 1:1 to about 1:1.5, about 1:1 to about 1:1.4, about 1:1 to about 1:1.3, about 1:1 to about 1:1.2, and about 1:1 to about 1:1.1. In embodiments described herein, the ratio of oil to polyol is about 1.2:1. In embodiments described herein, the ratio of oil to polyol is about 1:2.

In embodiments described herein, the percent oil in the oil:polyol formulation is from about 10% to about 60%. In embodiments described herein, the percent polyol in the oil:polyol formulation is from about 30% to about 70%.

In embodiments described herein, the ratio of oil to water is selected from the group consisting of about 1.1:1 to about 1:1.61, about 1:1 to about 1:1.5, about 1:1 to about 1:1.4, about 1:1 to about 1:1.3, about 1:1 to about 1:1.2, and about 1:1 to about 1:1.1. In embodiments described herein, the ratio of oil to water is about 1.1:1. In embodiments described herein, the ratio of oil to water is about 1:1.61.

In embodiments described herein, the percent oil in the oil:water formulation is from about 30% to about 70%. In embodiments described herein, the percent water in the oil:water formulation is from about 20% to about 60%.

In embodiments described herein, the ratio of castor oil to ethanol is selected from the group consisting of about 3:1 to about 1:3, about 2.9:1 to about 1:2.9, about 2.8:1 to about 1:2.8, about 2.7:1 to about 1:2.7, about 2.6:1 to about 1:2.6, about 2.5:1 to about 1:2.5, about 2.4:1 to about 1:2.4, about 2.3:1 to about 1:2.3, about 2.2:1 to about 1:2.2, about 2.1:1 to about 1:2.1, about 2:1 to about 1:2, about 1.9:1 to about 1:1.9, about 1.8:1 to about 1:1.8, about 1.7:1 to about 1:1.7, about 1.6:1 to about 1:1.6, about 1.5:1 to about 1:1.5, about 1.4:1 to about 1:1.4, about 1.3:1 to about 1:1.3, about 1.2:1 to about 1:1.2, about 1.1:1 to about 1:1.1, and about 1:1. In embodiments described herein, the ratio of castor oil to ethanol is about 1:2. In embodiments described herein, the ratio of castor oil to ethanol is about 1.45:1. In embodiments described herein, the ratio of castor oil to ethanol is about 2.45:1. In embodiments described herein, the ratio of castor oil to ethanol is about 1.2:1.

In embodiments described herein, the percentage of Phase B in the biphasic topical composition is selected from the group consisting of about 25% to about 80%, about 27% to about 75%, about 30% to about 70%, about 31% to about 65%, about 32% to about 60%, about 33% to about 60%, about 40% to about 90%, about 45% to about 85%, about 50% to about 80%, about 55% to about 75%, about 60% to about 70%, and about 65% to about 70%. In embodiments described herein, the percentage of Phase B in the biphasic topical composition is about 45% to about 65%. In embodiments described herein, the percentage of Phase B in the biphasic topical composition is about 45%. In embodiments described herein, the percentage of Phase B in the biphasic topical composition is about 47.5%. In embodiments described herein, the percentage of Phase B in the biphasic topical composition is about 50%. In embodiments described herein, the percentage of Phase B in the biphasic topical composition is about 53.5%. In embodiments described herein, the percentage of Phase B in the biphasic topical composition is about 54.5%. In embodiments described herein, the percentage of Phase B in the biphasic topical composition is about 56.34%. In embodiments described herein, the percentage of Phase B in the biphasic topical composition is about 57%. In embodiments described herein, the percentage of Phase B in the biphasic topical composition is about 62%.

In embodiments described herein, castor oil is in the amount selected from the group consisting of about 2.0% to about 15% in the final composition, about 2.5% to about 12.7% in the final composition, about 2.5% to about 14.5% in the final composition, about 3.0% to about 14% in the final composition, about 3.5% to about 13.5% in the final composition, about 4.0% to about 13% in the final composition, about 4.5% to about 12.5% in the final composition, about 5.0% to about 12% in the final composition, about 5.5% to about 11.5% in the final composition, about 6.0% to about 11% in the final composition, about 6.5% to about 10.5% in the final composition, about 7.0% to about 10% in the final composition, and about 7.5% to about 9.5% in the final composition. In embodiments described herein, castor oil is in the amount of about 2.5%. In embodiments described herein, castor oil is in the amount of about 6.0%. In embodiments described herein, castor oil is in the amount of about 12.7%.

In embodiments described herein, total amount of non-polar solvents in the composition is from about 14% to about 54%. In embodiments described herein, the non-polar solvents are selected from the group consisting of castor oil, isohexadecane, ethyl linoleate, C12-15 alkyl benzoate, caprylic/capric triglycerides, caprylyl methicone, phenyltrimethicone, Argan oil, Karanja oil, Karanja ester (Karanjate), Almond oil, CBD Oil, Coconut Oil, Sunflower Oil, Safflower oil, Hemp Oil, Jojoba Oil, Macadamia Nut Oil, and combinations thereof. In embodiments described herein, isohexane in an amount of about 10% to about 25%. In embodiments described herein, ethyl linoleate in an amount of about 2% to about 10%. In embodiments described herein, C12-15 alkyl benzoate in an amount of about 5% to about 16%. In embodiments described herein, caprylic/capric triglycerides in an amount of about 0.1% to about 3%.

In embodiments described herein, Phase A further comprises oil soluble ingredients or oil soluble active ingredients. The oil soluble ingredients or active ingredients dissolved in Phase A are selected from the group consisting of hydrophobically modified ascorbic acid, salicylic acid, phenethyl resorcinol or derivatives thereof, vitamin E acetate, resveratrol or derivatives thereof, derivatives of hydroxy cinnamic acids, organic sunscreens, hydrophobic-soluble natural extracts, retinoid derivatives, oil-soluble pharmaceutical derivatives, oil-soluble vitamins, oil-soluble skin lightening ingredients, anti-acne ingredients, anti-ageing ingredients, sunless tanning ingredients, peptides, antioxidants, amino acids, soothing ingredients, and combinations thereof. In embodiments described herein, the total amount of oil soluble ingredients in the composition is from about 0.001% to about 20%.

In embodiments described herein, ethanol is in the amount selected from the group consisting of about 2.0% to about 15% in the final composition, about 2.5% to about 14.5% in the final composition, about 3.0% to about 14% in the final composition, about 3.5% to about 13.5% in the final composition, about 4.0% to about 13% in the final composition, about 4.5% to about 12.5% in the final composition, about 5.0% to about 12% in the final composition, about 5.0% to about 8.74% in the final composition, about 5.5% to about 11.5% in the final composition, about 6.0% to about 11% in the final composition, about 6.5% to about 10.5% in the final composition, about 7.0% to about 10% in the final composition, and about 7.5% to about 9.5% in the final composition. In embodiments described herein, ethanol is in the amount of about 5.0%. In embodiments described herein, ethanol is in the amount of about 8.74%.

In embodiments described herein, total amount of polar solvents in the composition is from about 10% to about 70%. In embodiments described herein, the polar solvents are selected from the group consisting of glycerin, butylene glycol, propylene glycol, 1,3-propanediol, caprylyl glycol, hexanediol, pentylene glycol, polyol, water, PEG-400, sugar cane alcohol, and combinations thereof. Glycerin in an amount of about 10% to about 40%. Butylene glycol in an amount of about 10% to about 40%. Pentylene glycol in an amount of about 5% to about 12%. Polyol in an amount of about 0.1% to about 50%. The polyol is selected from the group consisting of polyol erthritol, polyol isomalt, polyol lactitol, polyol maltitol, polyol mannitol, polyol sorbitol, and polyol xylitol. Water in an amount of about 0.1% to about 50%.

In embodiments described herein, Phase B is substantially free of water.

In embodiments described herein, Phase B further comprises water or polyol soluble ingredients or water or polyol soluble active ingredients. The water or polyol soluble ingredients or active ingredients dissolved in Phase B are selected from the group consisting of niacinamide, ascorbic acid, extracts, kojic acid, citric acid, benzoyl peroxide, carbamide peroxide, azelaic acid, mandelic acid or other alpha-hydroxy acids, allantoin, hyaluronic acid and derivatives thereof, carbohydrates, water/polyol soluble pharmaceuticals, DHA, erythrulose, and combinations thereof. Total amount of water or polyol soluble ingredients in the composition is from about 0.5% to about 20%.

In embodiments described herein, the biphasic topical composition comprising: a Phase A including castor oil, one or more additional non-polar solvents, and one or more oil soluble active ingredients, and a Phase B including ethanol, one or more additional polar solvents, and one or more polar or water soluble active ingredients, wherein the ratio of castor oil to ethanol is about 1:1 to about 3:1; and wherein the biphasic topical composition is free of surfactants and emulsifiers.

In embodiments described herein, the biphasic topical composition comprising: a Phase A including castor oil, one or more additional non-polar solvents, and one or more oil soluble active ingredients, and a Phase B including ethanol, one or more additional polar solvents, and one or more polar or water soluble active ingredients, wherein the ratio of castor oil to ethanol is about 1:1 to about 3:1; and wherein the biphasic topical composition is free of surfactants and emulsifiers. In embodiments described herein, the one or more oil soluble active ingredients is phenethyl resorcinol, the one or more polar or water soluble active ingredients is active L-ascorbic acid, and the biphasic topical composition is used to treat uneven skin tone.

In embodiments described herein, the biphasic topical composition comprising: a Phase A including castor oil, one or more additional non-polar solvents, and one or more oil soluble active ingredients, and a Phase B including ethanol, one or more additional polar solvents, and one or more polar or water soluble active ingredients, wherein the ratio of castor oil to ethanol is about 1:1 to about 3:1; and wherein the biphasic topical composition is free of surfactants and emulsifiers. In embodiments described herein, the one or more oil soluble active ingredients is salicylic acid, the one or more polar or water soluble active ingredients are selected from active L-ascorbic acid, citric acid, niacinamide, or combinations thereof, and the biphasic topical composition is used to treat acne. In embodiments, the citric acid is at about 10%.

In embodiments described herein, the biphasic topical composition comprising: a Phase A including castor oil, one or more additional non-polar solvents, and one or more oil soluble active ingredients, and a Phase B including ethanol, one or more additional polar solvents, and one or more polar or water soluble active ingredients, wherein the ratio of castor oil to ethanol is about 2:1 to about 1:3; and wherein the biphasic topical composition is free of surfactants and emulsifiers. In embodiments described herein, the one or more oil soluble active ingredients are selected from tetrahexyldecyl, ascorbate, or combinations thereof, the one or more polar or water soluble active ingredients is niacinamide, and the biphasic topical composition is used to lighten or brighten the skin.

In embodiments described herein, the biphasic topical composition comprising: a Phase A including castor oil, one or more additional non-polar solvents, and one or more oil soluble active ingredients, and a Phase B including ethanol, one or more additional polar solvents, and one or more polar or water soluble active ingredients, wherein the ratio of castor oil to ethanol is about 1:1 to about 3:1; and wherein the biphasic topical composition is free of surfactants and emulsifiers. In embodiments described herein, the one or more oil soluble active ingredients are selected from homosalate, ethylhexyl salicylate, octinoxate, octocrylene, butylmethoxydibenzoylmethane, or combinations thereof, the one or more polar or water soluble active ingredients is L-ascorbic acid, and the biphasic topical composition is used as a sunscreen. In embodiments, the L-ascorbic acid is at about 0.5% to about 15%. Concentrations of sunscreen actives in a Phase A would be selected based on regional regulations (Global, USA, EU, Japan, China, Mercosur, Australia).

In embodiments described herein, the biphasic topical composition comprising: a Phase A including castor oil, one or more additional non-polar solvents, and one or more oil soluble active ingredients, and a Phase B including ethanol, one or more additional polar solvents, and one or more polar or water soluble active ingredients, wherein the ratio of castor oil to ethanol is about 1:1 to about 3:1; and wherein the biphasic topical composition is free of surfactants and emulsifiers. In embodiments described herein, the one or more oil soluble active ingredients are selected from homosalate, ethylhexyl salicylate, butylmethoxydibenzoylmethane, or combinations thereof, the one or more polar or water soluble active ingredients is niacinamide, and the biphasic topical composition is used in an antiaging regimen. In embodiments, the niacinamide is at about 1% to about 5%.

The biphasic topical compositions described herein are unstable and naturally separate into two phases, i.e. Phase A and Phase B. In embodiments described herein, the time of separation of Phase A and Phase B after mixing is between about 10 seconds to about 5 minutes.

In some embodiments, the biphasic topical composition further comprises a pharmaceutically acceptable excipient.

In some embodiments, the biphasic topical composition further comprises a pharmaceutical additive, a cosmetic additive, an additional agent, or combinations thereof. In some embodiments, the biphasic topical composition further comprises both a pharmaceutical additive and a cosmetic additive. In some embodiments, the pharmaceutical additive, the cosmetic additive, the additional additives, or combination thereof are in a total amount of at least about 2.0 wt %. In some embodiments, the pharmaceutical additive, the cosmetic additive, the additional additive, or combination thereof are in a total amount selected from the group consisting of about 2 wt % to about 50 wt %, about 2 wt % to about 45 wt %, about 2 wt % to about 40 wt %, about 2 wt % to about 35 wt %, about 2 wt % to about 30 wt %, about 2 wt % to about 25 wt %, about 2 wt % to about 20 wt %, about 2 wt % to about 15 wt %, about 2 wt % to about 10 wt %, about 2 wt % to about 5 wt %, about 2 wt % to about 4 wt %, and about 2 wt % to about 3 wt %.

In some embodiments, the pharmaceutical additive is selected from the group consisting of diluents, fillers, disintegrants, binders, lubricants, surfactants, hydrophobic vehicles, water soluble vehicles, emulsifiers, buffers, humectants, moisturizers, solubilizers, preservatives, colorants, plastizers, carriers, excipients, film formers, and combinations thereof. In some embodiments, the pharmaceutical additive is a pharmaceutical agent selected from the group consisting of antihistamine, analgesic, rash treatment agents, anti-itch agents, and combinations thereof. The person of ordinary skill in the art can refer to various pharmacologic references such as, for example, Modern Pharmaceutics, Banker & Rhodes, Marcel Dekker, Inc. (1979) and Goodman & Gilman's The Pharmaceutical Basis of Therapeutics, 6th Edition, MacMillan Publishing Co, New York (1980) for guidance in determining the amount of such components in the compositions and formulations of embodiments.

In some embodiments, the cosmetic additive is selected from the group consisting of vitamins, cosmetic peptides, oil control agents, sensation modifying agents, skin lightening agents, hydrating formulations, sunscreen agents, compounds that absorbs or reflects UV photons, other skin care agents, and combinations thereof.

In some embodiments, the additional additive is selected from the group consisting of hydroxyacetophenone, sodium phytate, caprylic/capric triglyceride, sodium acrylates copolymer, octyldodecanol, octyldodecyl xyloside, PEG-30 dipolyhydroxystearate, Jojoba esters, Helianthus annuus (sunflower) seed wax, Acacia decurrens flower wax, polyglycerin-3, acrylamide/sodium acryloyldimethyl taurate copolymer, isohexadecane, polysorbate 80, cyclopentasiloxane, dimethicone/vinyl dimethicone crosspolymer, ethylhexylglycerin, and combinations thereof.

In some embodiments, the biphasic topical composition may further comprise abrasives, antiacne agents, antidandruff agents, antifungal agents, antimicrobial agents, antioxidants, toners, moisturizers, skin conditioning agents, humectants, emollients, occlusive agents, skin bleaching or lightening agents, proteins, cleaners, hair conditioners, and the like.

Abrasives may be used to remove unwanted skin such as dead skin cells and calluses. In some embodiments, the abrasive is selected from the group consisting of alumina, aluminum silicate, apricot seed powder, attapulgite, avocado powder, bamboo powder, barley flour, bentonite, calcium carbonate, calcium phosphate, calcium pyrophosphate, calcium sulfate, chalk, chitin, coconut shell powder, colloidal oatmeal, comfrey leaf powder, corn cob meal or powder, corn flour, corn meal, corn starch, diamond powder, diatomaceous earth, dicalcium phosphate, dicalcium phosphate dehydrate, egg shell powder, Fuller's earth, hydrated silica, hydroxyapatite, kaolin, kiwi seed, lauryl acrylate polymers, loess, magnesium potassium fluorosilicate, magnesium trisilicate, microcrystalline cellulose, montmorillonite, Moroccan lava clay, oat bran, oat flour, oatmeal, oyster shell powder, peach pit powder, peanut flour, pecan shell powder, polyethylene, pumice, raspberry seed, rice bran, rye flour, sand, silica, sodium bicarbonate, sodium hydroxypropyl starch phosphate, sodium magnesium fluorosilicate, sodium silicoaluminate, soybean flour, sweet almond meal, talc, tin oxide, tricalcium phosphate, walnut shell powder, wheat bran, wheat flour, wheat powder, wheat starch, wood powder, zirconium silicate, and derivatives and combinations thereof.

The active ingredients that may be included within the appropriate phase of the biphasic topical composition are anti-acne agents, antidandruff agents, antimicrobial agents, antifungal agents, antioxidants, toners, skin conditioning or moisturizing agents, skin bleaching or lightening agents, hair conditioners, proteins, cleansers, oil control agents, skin care agents, anti-aging ingredients, sunscreen agents, sensation modifying agents, cooling agents, warming agents, relaxing or soothing agents, stimulating or refreshing agent, anti-itch ingredients, bug repellant ingredients, and combinations thereof.

Anti-acne agents may be used to treat blemishes, pimples, blackheads, and whiteheads. In some embodiments, the anti-acne agent is selected from the group consisting of salicylic acid, benzoyl peroxide, carbamide peroxide, glycolic acid, alpha-hydroxy acids, retinal, retinol, retinaldehyde, vitamin A, vitamin A derivative, azelaic acid, mandelic acid, niacinamide, willow bark extract, licorice root extract, citric acid, or sulfur, and their derivatives and combinations thereof. In certain embodiments, the anti-acne agent is part of Phase A and is selected from the group consisting of salicylic acid, retinoids such as retinol, retinal, hydroxy pinacolone retinoate, sulfur, tea tree oil, bakuchiol, bakutrol, honokiol, phloretin, and combinations thereof. In certain embodiments, the anti-acne agent is part of Phase B and is selected from the group consisting of active L-ascorbic acid, caprylol glycine, undecylenoyl glycine, Phytosphingosine, peroxides, mandelic acid, and combinations thereof.

Antidandruff agents may be used to treat dandruff, seborrheic dermatitis, or psoriasis. In some embodiments, the antidandruff agent is selected from the group consisting of coal tar, salicylic acid, selenium sulfide, sulfur, zinc pyrithione, capryloyl glycine, and their derivatives and combinations thereof.

Antifungal agents include agents that inhibit the growth and reproduction of fungal cells or decreases the number of fungi present. In some embodiments, the antifungal agent is selected from the group consisting of calcium undecylenate, ketoconazol, povidone-iodine (PVP-iodine), tea tree oil, tasmanian native pepper, undecylenic acid, zinc undecylenate, and their derivatives and combinations thereof.

Antimicrobial agents include agents that kill microorganisms, prevent or inhibit microorganism growth and reproduction, or agents that help prevent infection in minor cuts, scrapes, and burns. In some embodiments, the antimicrobial agent is selected from the group consisting of lower chain (C1-C4) alcohols, quaternary ammonium compounds such as benzalkonium chloride and benzethonium chloride, clindamycin, methylbenzethonium chloride, hydrogen peroxide, Oligopeptide-10, phenols, tea tree oil, triclosan, povidone-iodine (PVP-Iodine), and their derivatives and combinations thereof.

Antioxidants include agents that are characterized as free radical scavengers and help reverse skin damage caused by free radicals. In some embodiments, the antioxidant is selected from the group consisting of acetyl cysteine, alpha lipoic acid, arbutin, ascorbic acid (vitamin C), ascorbic acid polypeptide, ascorbyl dipalmitate, ascorbyl methylsilanol pectinate, ascorbyl palmitate, ascorbyl stearate, BHA, BHT, t-butyl hydroquinone, caffeic acid, Camellia sinensis oil, carotenoids, chitosan ascorbate, chitosan glycolate, chitosan salicylate, chlorogenic acids, CoQ10, cortisen, cysteine, cysteine HC1, decyl mercaptomethylimidazole, diamylhydroquinone, di-t-butylhydroquinone, dicetyl thiodipropionate, dicyclopentadiene/t-butylcresol copolymer, digalloyl trioleate, dilauryl thiodipropionate, dimyristyl thiodipropionate, dioleyl tocopheryl methylsilanol, diosmine, disodium ascorbyl sulfate, disodium rutinyl disulfate, distearyl thiodipropionate, ditridecyl thiodipropionate, dodecyl gallate, dunaliella salina extract, erythorbic acid, ethyl ferulate, ferulic acid, hydroquinone, p-hydroxyanisole, hydroxyl amine HC1, hydroxyl amine sulfate, hydroxytyrosol, isooctyl thioglycolate, isoquercitrin, kojic acid, madecassicoside, magnesium ascorbate, magnesium ascorbyl phosphate, melatonin, methoxy-PEG-7 rutinyl succinate, methylene di-t-butylcresol, methylsilanol ascorbate, nordihydroguaiaretic acid, octyl gallate, phenylthioglycloic acid, phloroglucinol, potassium ascorbyl tocopheryl phosphate, potassium sulfite, propyl gallate, resveratrol, rosmarinic acid, rutin, sirtunis, sodium ascorbate, sodium ascorbyl/cholesteryl phosphate, sodium bisulfite, sodium erythorbate, sodium metabisulfite, sodium sulfite, sodium thioglycolate, sorbityl furfural, tea tree oil, tetrahexyldecyl ascorbate, tetrahydrodiferuloylmethane, thiodiglycol, thiodiglycolamide, thiodiglycolic acid, thioglycolic acid, thiolactic acid, thiosalicylic acid, thiotaurine, tocophereth derivatives, tocopherol (vitamin E), tocophersolan, tocopheryl acetate, tocopheryl linoleate, tocopherol linoleate/oleate, tocopheryl nicotinate, tocopheryl succinate, tocoquinone, o-tolyl biguanide, tri(nonylphenyl)phosphate, ubiquinone, vitamin D, zinc dibutyldithiocarbamate, and their derivatives and combinations thereof.

Toners include agents that create a tightening or tingling sensation on skin. In some embodiments, the toner is selected from the group consisting of ammonium alum, calcium chloride, calcium lactate, dimethyl MEA, gallic acid, lens esculenta (lentil) seed extract, potassium alum, sodium alum, sodium aluminum chlorohydroxy lactate, sodium aluminum lactate, tannic acid, tioxolone, tranexamic acid, zinc acetate, zinc chloride, zinc lactate, zinc phenolsulfonate, zinc sulfate, zirconium chlorohydrate, witch hazel, alcohol derivatives such as denatured alcohol and SD alcohol, aluminum derivatives such as aluminum acetate, aluminum bromohydrate, aluminum chloride, aluminum chlorohydrex, aluminum citrate, aluminum diacetate, aluminum dichlorohydrate, aluminum dichlorohydrex, aluminum glycinate, aluminum lactate, aluminum phenolsulfonate, aluminum sesquichlorohydrate, aluminum sesquichlorohydrex, and aluminum sulfate, aluminum zirconium derivatives such as aluminum zirconium octachlorohydrex, aluminum zirconium pentachlorohydrate, aluminum zirconium pentachlorohydrex, aluminum zirconium tetrachlorohydrate, aluminum zirconium tetrachlorohydrex, aluminum zirconium trichlorhydrate, and aluminum zirconium trichlorohydrex, and their derivatives and combinations thereof.

Skin conditioning agents or moisturizers can be classified into different groups such as emollients, humectants, and occlusive agents. Emollients include agents that remain on the upper layers of skin and act as lubricants and improve appearance. In some embodiments, the emollient is selected from the group consisting of petrolatum, petrolatum plus volatile silicones, cold cream (USP), hydrophilic ointment (USP), lanolin, glycerides, fruit oils, nut oils, vegetable oils, dimethicones, methicone, cyclomethicone, dormin, fatty acids, myristate derivatives like butyl myristate and myristyl myristate, oleate derivates, C1-C4 glycols, fatty acid glycols, glycol esters, glycerine, glycerols, paraffin, rapeseed oil, long chain alcohols, olive oil, jojoba oil, castor oil, and their derivatives and combinations thereof. Humectants include agents that increase the water content of the top layer of skin. In some embodiments, the humectant is selected from the group consisting of allantoin, agarose, arginine, benzyl hyaluronate, chitosan, copper, corn glycerides, gluconolactone, lactic acid, lactobionic acid, lactose, lysine, kombucha, maltitol, maltose, mannitol, propylene glycol, butylene glycol, pentylene glycol, propanediol, sodium aspartate, fructose, honey, glycerin, diglycerin, betaine, diols, urea derivatives, 1,2-hexanediol, D-ribose, glucose, sorbitol, dextrose, urea, 2-Pyrrolidone-5-Carboxylic Acid and related salts, sea salt, inorganic salts of citric acid, inorganic salts of lactic acid, ectoin, glycolic acid, and their derivatives and combinations thereof. Occlusive agents slow the evaporation of water from skin. In some embodiments, the occlusive agent is selected from the group consisting of petrolatum, shea butter, dimethicones, plant and animal oils such as avocado, canola, cod liver, and corn, mineral oil, olive oil, soybean oil, lanolin, glycerides, beeswax, triglycerides, long chain fatty alcohols, coco butter, coconut oil, jojoba oil, propylene glycol and their derivatives and combinations thereof.

In addition to skin conditioning agents that provide a moisturizing benefit, there are other skin conditioning agents that improve the appearance of skin. In some embodiments, the skin conditioning agent is selected from the group consisting of cholesterol, cystine, hyaluronic acid, keratin, egg yolk, glycine, gluconolactone, lactic acid, lactobionic acid, panthenol, retinol, salicylic acid, vegetable oil, proteins, vitamins, bisabolol, ceramide, coenzyme A, lecithin and their derivatives and combinations thereof. In certain embodiments, the skin conditioning agent is part of Phase A and is selected from the group consisting of karanja oil, karanja ester (Karanjate), almond oil, CBD Oil, Coconut Oil, Sunflower Oil, Safflower oil, Hemp Oil, Jojoba Oil, Macadamia Nut Oil, coated Iron Oxides, Titanium dioxide compatible with oil phase, and combinations thereof. In certain embodiments, the skin conditioning agent is part of Phase B and is selected from the group consisting of niacinamide, peptides, amino acids, and combinations thereof. In certain embodiments, the skin conditioning agent is part of Phase A and is selected from the group consisting of ascorbic acid derivatives, oil soluble vitamins, urea derivatives, and combinations thereof. In certain embodiments, the skin conditioning agent is part of Phase B and is selected from the group consisting of vitamin B3 compounds, niacinamide, azelaic acid, butyl hydroxyanisole, gallic acid and its derivatives, glycyrrhizinic acid salts, hydroquinone, kojic acid, arbutin, deoxyarbutin, ferulic acid, ferulic acid dimer, mulberry extract, Ovaliss ((S)-5,6,6a,7-Tetrahydro-1,2,9,10-tetramethoxy-6-methyl-4H-dibenzo[de,g] quinoline, D-Glucopyranose, oligomeric, C10-16-alkyl glycosides, water, ethyl alcohol, and glycerin, Whey protein, MPC (Milk protein complex), Sesaflash (Glycerin, Acrylates copolymer, PVP/polycarbamyl polyglycol ester, Hydrolyzed Sesame Protein PG—propyl methylsilanediol), Majestem (Glycerin, Leontopodium Alpinum Callus Culture Extract and xanthan gum), Idealift (butylene glycol, sorbitan laurate, hydroxyethylcellulose, acetyl dipeptide-1 cetyl ester), and combinations thereof.

Skin bleaching or lightening agents include agents that lighten pigment in skin. The preferred skin bleaching agent is hydroquinone. In some embodiments, the brightener is selected from the group consisting of azelaic acid, bearberry extract, arbutin, deoxyarbutin, Glycyrrhiza glabra (Licorice) root extract, kojic acid, peat extract, and their derivatives and combinations thereof. In certain embodiments, the skin bleaching or lightening agent for treating uneven skin tone is part of Phase A and is selected from the group consisting of phenethyl resorcinol, hydroquinone, kojic acid, kojic acid esters deoxyarbutin and all derivatives thereof, sclareolide (under trade name SymBright), retinoids such as retinol, hydroxy pinacolone retinoate, retinal, mequinol, N-acetyl-4-S-Cysteaminylphenol, azelaic acid, cysteamine, mandelic acid, gentisic acid, flavonoids, aloesin, tetrahydrodiferuloylmethane, and combinations thereof. In certain embodiments, the skin bleaching or lightening agent for treating uneven skin tone is part of Phase B and is selected from the group consisting of active L-ascorbic acid, niacinamide, licorice root extracts, papaya extracts, ellagic acid, tranexamic acid, papain, arbutin, peptides (such as tetrapeptide-30), and combinations thereof. In certain embodiments, the lightening or brightening agent is part of Phase A and is selected from the group consisting of urea derivatives, sclareolide, retinoids, and combinations thereof. In certain embodiments, the lightening or brightening agent is part of Phase B and is selected from the group consisting of azelaic acid, bearberry extract, arbutin, deoxyarbutin, Glycyrrhiza glabra (Licorice) root extract, kojic acid, peat extract, niacinamide, ascorbic acid, Centella asiatica extract, citric acid, benzoyl peroxide, carbamide peroxide, mandelic acid or other alpha-hydroxy acids, and their derivatives and combinations thereof.

Hair conditioners include agents that enhance the appearance and feel of hair by improving a property like gloss, texture, or body. In some embodiments, the hair conditioner is selected from the group consisting of lanolin, silicone, dimethicone, proteins such as amino acids, collagen, and keratin, vitamins, betaine surfactants, amine oxide surfactants, ceramide, fatty acids, eggs, milk, natural plant and animal oils, mineral oil, olive oil, polyquaternium, and their derivatives and combinations thereof. In some embodiments, the hair conditioner is a hair color or anti-greying agent. In certain embodiments, the hair conditioning agent is part of Phase A and is selected from the group consisting of lanolin, silicone, dimethicone, ceramide, fatty acids, natural plant and animal oils, mineral oil, olive oil, argan oil, oil-soluble vitamins, and combinations thereof. In certain embodiments, the hair conditioning agent is part of Phase B and is selected from the group consisting of proteins, amino acids, collagen, and keratin, vitamins, betaine surfactants, amine oxide surfactants, eggs, milk, polyquaternium, In some embodiments, the protein is selected from the group consisting of collagen, keratin, soy protein, wheat protein, bean palmitate, ascorbic acid polypeptide, the amino acids, casein, cholecalciferol polypeptide, rice protein, silk protein, gluten protein, lysine, acetyl glucosamine, actin, actizyme, albumen, conchiorin protein, corn protein, egg protein, elastin, fibronectin, gadidae protein, hemoglobin, hexapeptide-21, lactalalbumin, lupine protein, maple sycamore protein, milk protein, myristoyl pentapeptide-8, myristoyl tetrapeptide-8, oat protein, oligopeptide 10, palmitoyl hexapeptide-14, palmitoyl oligopeptide, palmitoyl tetrapeptide-7, pea protein, potato protein, reticulin, rice bran protein, serum protein, sweet almond protein, tetrapeptide-16, vegetable protein, yeast protein, palmitoyl oligopeptide, pantothenic acid polypeptides, milk solids, sericin, albumen, amylase, amyloglucosidase, arginine, bromelain, catalase, gelatin, zein, crystallins, cytochrome C, deoxyribonuclease, gliadin, glucose oxidase, glycoproteins, lactoferrin, lactoglubulin, lactoperoxidase, lipase, nisin, oxido reductases, papain, pepsin, subtilisin, sutilains, and their derivatives and combinations thereof. In certain embodiments, the hair conditioning agent is part of Phase A is selected from the group consisting of argan oil, olive oil, karanja oil, karanja ester (karanjate), ceramides, and combinations thereof. In certain embodiments, the hair conditioning agent is part of Phase B is selected from the group consisting of panthenol, amino acids, plant extracts, peptides, and combinations thereof.

Proteins include animal, plant, fungi, yeast, and bacteria proteins that have skin health benefits. In some embodiments, the protein is selected from the group consisting of collagen, keratin, soy protein, wheat protein, bean palmitate, ascorbic acid polypeptide, the amino acids, casein, cholecalciferol polypeptide, rice protein, silk protein, gluten protein, lysine, acetyl glucosamine, actin, actizyme, albumen, conchiorin protein, corn protein, egg protein, elastin, fibronectin, gadidae protein, hemoglobin, hexapeptide-21, lactalalbumin, lupine protein, maple sycamore protein, milk protein, myristoyl pentapeptide-8, myristoyl tetrapeptide-8, oat protein, oligopeptide 10, palmitoyl hexapeptide-14, palmitoyl oligopeptide, palmitoyl tetrapeptide-7, pea protein, potato protein, reticulin, rice bran protein, serum protein, sweet almond protein, tetrapeptide-16, vegetable protein, yeast protein, palmitoyl oligopeptide, pantothenic acid polypeptides, milk solids, sericin, albumen, amylase, amyloglucosidase, arginine, bromelain, catalase, gelatin, zein, crystallins, cytochrome C, deoxyribonuclease, gliadin, glucose oxidase, glycoproteins, lactoferrin, lactoglubulin, lactoperoxidase, lipase, nisin, oxido reductases, papain, pepsin, subtilisin, sutilains, and their derivatives and combinations thereof.

Cleansers include agents that are used for cleaning the skin and hair by solubilizing oil and suspending soils. Cleansers may be foaming or non-foaming. Exemplary cleaners are typically a surfactant and can be characterized as nonionic, anionic, or zwitterionic. In some embodiments, the cleanser is selected from the group consisting of taurates, sulfates, sulfonates, carboxylates, sulfosuccinates, sarcosinates, zwitterionic betaines, fatty acid and fatty alcohol derivatives, and alkylpolyglucoside and amine oxide surfactants. In some embodiments, the cleansers may be combined with some abrasives such as clays and sulfurs to provide light exfoliation.

In some embodiments, the biphasic topical composition may further comprise an oil control agents. Oil control agents are compounds useful for regulating the production of skin oil, or sebum, and for improving the appearance of oily skin. In some embodiments, the oil control agent is selected from the group consisting of salicylic acid, dehydroacetic acid, benzoyl peroxide, vitamin B3 (for example, niacinamide), and the like, their isomers, esters, salts and derivatives, and combinations thereof.

In some embodiments, the biphasic topical composition may further comprise other skin care agents selected from the group consisting of retinol, steroids, sunblock, salicylate, minocycline, antifungals, peptides, antibodies, lidocaine, and the like and combinations thereof. In some embodiments, other skin care agents include N-acyl amino acid compounds comprising, for example, N-acyl phenylalanine, N-acyl tyrosine, and the like, their isomers, comprising their D and L isomers, salts, derivatives, and mixtures thereof. An example of a suitable N-acyl amino acid is N-undecylenoyl-L-phenylalanine is commercially available under the tradename SEPIWHITE®. Other skin active agents include, but are not limited to, Lavandox, Thallasine 2, Argireline NP, Gatuline In-Tense and Gatuline Expression, Myoxinol LS 9736, Syn-ake, and Instensyl®, Sesaflash™, N-acetyl D-glucosamine, panthenol (for example, DL panthenol available from Alps Pharmaceutical Inc.), tocopheryl nicotinate, benzoyl peroxide, 3-hydroxy benzoic acid, flavonoids (for example, flavanone, chalcone), farnesol, phytantriol, glycolic acid, lactic acid, 4-hydroxy benzoic acid, acetyl salicylic acid, 2-hydroxybutanoic acid, 2-hydroxypentanoic acid, 2-hydroxyhexanoic acid, cis-retinoic acid, trans-retinoic acid, retinol, retinyl esters (for example, retinyl propionate), phytic acid, N-acetyl-L-cysteine, lipoic acid, tocopherol and its esters (for example, tocopheryl acetate: DL-a-tocopheryl acetate available from Eisai), azelaic acid, arachidonic acid, tetracycline, ibuprofen, naproxen, ketoprofen, hydrocortisone, acetominophen, resorcinol, phenoxyethanol, phenoxypropanol, phenoxyisopropanol, 2,4,4′-trichloro-2′-hydroxy diphenyl ether, 3,4,4′-trichlorocarbanilide, octopirox, lidocaine hydrochloride, clotrimazole, miconazole, ketoconazole, neomycin sulfate, theophylline, and mixtures thereof. Further skin care agents are disclosed in US Publication No. 2007/0020220A1, wherein the components/ingredients are incorporated herein by reference in their entirety.

In some embodiments, the biphasic topical composition may further comprise antiaging ingredients, effective for skin or hair, selected from the group consisting of ascorbic acid and its derivative compounds, vitamin B3 compounds, azelaic acid, butyl hydroxyanisole, gallic acid and its derivatives, glycyrrhizinic acid, hydroquinone, kojic acid, arbutin, deoxyarbutin, ferulic acid, ferulic acid dimer, mulberry extract, and combinations thereof. In some embodiments, the topical composition or final formulation may comprise Ovaliss ((S)-5,6,6a,7-Tetrahydro-1,2,9,10-tetramethoxy-6-methyl-4H-dibenzo[de,g] quinoline, 1,2-Octanediol, D-Glucopyranose, oligomeric, C10-16-alkyl glycosides, water, ethyl alcohol, and glycerin), Whey protein, MPC (Milk protein complex), Sesaflash (Glycerin, Acrylates copolymer, PVP/polycarbamyl polyglycol ester, Hydrolyzed Sesame Protein PG—propyl methylsilanediol), Majestem (glycerin, Leontopodium Alpinum Callus Culture Extract and xanthan gum), or Idealift (butylene glycol, sorbitan laurate, hydroxyethylcellulose, and acetyl dipeptide-1 cetyl ester). In certain embodiments, the antiaging ingredient is part of Phase A and is selected from the group consisting of retinoids, oil-soluble vitamins and their derivatives, multifunctional oils, e.g. Karanja oil, Karanja ester (karanjate), Almond oil, CBD Oil, Coconut Oil, Sunflower Oil, Safflower oil, Hemp Oil, Jojoba Oil, Macadamia Nut Oil, and combinations thereof. In certain embodiments, the antiaging ingredient is part of Phase B and is selected from the group consisting of niacinamide, vitamin C, botanical plant extracts, amino acids, peptides, plant growth factors, urea derivatives, and combinations thereof.

In some embodiments, the biphasic topical composition may further comprises sunscreen agents selected from the group consisting of para-aminobenzoic acid (PABA), PABA esters (glyceryl PABA, amyldimethyl PABA and octyldimethyl PABA), benzophenones (oxybenzone and sulisobenzone), cinnamates (octylmethoxy cinnamate and cinoxate), salicylates (homomethyl salicylate) anthranilates, TiO₂, avobenzone, bemotrizinol, bisoctrizole, 3-(4-methylbenzylidene)-camphor, cinoxate, diethylamino hydroxybenzoyl hexyl benzoate, dioxybenzone, drometrizole trisiloxane, ecamsule, ethylhexyl triazone, homosalate, menthyl anthranilate, octocrylene, octyl salicylate, iscotrizinol, isopentenyl-4-methoxycinnamate, octyl-dimethyl-p-aminobenzoic acid, octyl-methoxycinnamate, oxybenzone, polysilicone-15, trolamine salicylate, ZnO, and combinations thereof. In certain embodiments, the sunscreen agent is part of Phase A and is selected from the group consisting of Homosalate, Ethylhexyl Salicylate, Octinozate, Octocrylene, Butyl Methoxydibenzoylmethane, ZnO, TiO2, bi s-Ethylhexyloxyphenol Methoxyphenyl Triazine (Tinosorb® S), Diethyl amino Hydroxybenzoyl Hexyl Benzoate (Uvinul® A Plus), Drometrizole Trisiloxane, Menthyl Anthranilate, Zinc Oxide (nano), lipophilic phase (Z-Cote®), Zinc Oxide (nano), lipophilic phase (Z-Cote® HP1), 4-Methylbenzylidene Camphor, Benzophenone-3, Benzophenone-4 (Uvinul® MS 40), Diethylhexyl Butamido Triazone, Ethylhexyl Methoxycinnamate (Uvinul® MC 80), Ethylhexyl Triazone (Uvinul® T 150), Ethylhexyl dimethyl PABA, Homomenthyl Salicylate, Isoamyl p-Methoxycinnamate, Octocrylene (Uvinul® N 539 T), PEG-25 PABA, Polysilicone-15, Titanium Dioxide (nano), hydrophilic phase, Tris Biphenyl Triazine (nano), (Tinosorb® A2B), and combinations thereof. In certain embodiments, the sunscreen agent is part of Phase B and is selected from the group consisting of Bis-Ethylhexyloxyphenol Methoxyphenyl Triazine, (Tinosorb® S Lite Aqua), Methylene Bis-Benzotriazolyl Tetramethylbutylphenol (nano) (Tinosorb® M), Phenylbenzimidazol Sulfonic Acid, Terephthalylidene Dicamphor Sulfonic Acid, Disodium Phenyl Dibenzimidazole Tetrasulfonate, Titanium Dioxide (nano) hydrophilic, Zinc Oxide (nano) hydrophilic, and combinations thereof.

In some embodiments, the biphasic topical composition may comprise a sensory modifying agent selected from the group of a cooling agent, a warming agent, a relaxing or soothing agent, a stimulating or refreshing agent, and combinations thereof.

In some embodiments, the cooling agent is selected from the group consisting of menthol; an isomer of menthol, a menthol derivative; 4-Methyl-3-(1-pyrrolidinyl)-2[5H]-furanone; WS-23, Icilin, Icilin Unilever Analog, 5-methyl-4-(1-pyrrolidinyl)-3-[2H]-furanone; 4,5-dimethyl-3-(1-pyrrolidinyl)-2 [5H]-furanone; isopulegol, 3-(1-menthoxy)propane-1,2-diol, 3-(1-menthoxy)-2-methylpropane-1,2-diol, p-menthane-2,3-diol, p-menthane-3,8-diol, 6-isopropyl-9-methyl-1,4-dioxas-piro[4,5]decane-2-methanol, menthyl succinate and its alkaline earth metal salts, trimethylcyclohexanol, N-ethyl-2-isopropyl-5-methylcyclohexanecarb-oxamide, Japanese mint (Mentha arvensis) oil, peppermint oil, menthone, menthone glycerol ketal, menthyl lactate, 3-(1-menthoxy)ethan-1-ol, 3-(1-menthoxy)propan-1-ol, 3-(1-menthoxy)butan-1-ol, 1-menthylacetic acid N-ethyl amide, 1-menthyl-4-hydroxypentanoate, 1-menthyl-3-hydroxybutyrate, N,2,3-trimethyl-2-(1-methylethyl)-butanamide, spearmint oil and combinations thereof.

In some embodiments, the warming agent is selected from the group consisting of polyhydric alcohols, capsaicin, capsicum powder, a capsicum tincture, capsicum extract, capsaicin, hamamelis, homocapsaicin, homodihydrocapsaicin, nonanoyl vanillyl amide, nonanoic acid vanillyl ether, vanillyl alcohol alkyl ether derivatives, such as vanillyl ethyl ether, vanillyl butyl ether, vanillyl pentyl ether, and vanillyl hexyl ether, isovanillyl alcohol alkyl ethers, ethylvanillyl alcohol alkyl ethers, veratryl alcohol derivatives, substituted benzyl alcohol derivatives, substituted benzyl alcohol alkyl ethers, vanillin propylene glycol acetal, ethylvanillin propylene glycol acetal, ginger extract, ginger oil, gingeol, gingeron, and combinations thereof.

In some embodiments, the relaxing or soothing agent is selected from the group consisting of herb extracts, aloe vera, alpha bisabolol, D-panthenol, allantoin, hamamelis, chamomile, yarrow; calendula, comfrey, witch hazel and other astringents, sea weed, and oat extracts; oils, selected from the group consisting of: almond oil, avocado oil, and comfrey; and essential oils, selected from the group consisting of: cardamone, eucalyptus, Mentha piperita (peppermint), hyssop, and rosemary; waxy or unctuous substances selected from the group consisting of: lanolin or vaseline jelly, minerals, selected from the group consisting of: zinc oxide, calamine and selenium; vitamins, selected from the group consisting of: tocopheryl acetate (vitamin E), and pharmaceutical agents selected from the group consisting of: analgesics, anesthetics, anti-inflammatory agents, and anti-histamines, and muscle relaxants; menthol, camphor, eugenol, eucalyptol, safrol, methyl salicylate, menthyl lactate, menthyl ethoxyacetate, menthone glycerinacetal, 3-1-menthoxypropane-1,2-diol, ethyl 1-menthyl carbonate, (1 S,3S,4R)-p-menth-8-en-3-ol, menthyl pyrrolidone carboxylate, N-substituted-p-menthane-3-carboxamides hamamelis extract, ginger oil, and combinations thereof.

In some embodiments, the stimulating or refreshing agent is selected from the group consisting of alcohol, L-menthol, camphor, menthe oil, capsicum extract, capsaicin, benzyl nicotinate, salicylate, glycol salicylate, acetyl choline, serotonin, histamine, a prostaglandin, a neurotransmitter, a CNS stimulant, caffeine, quinine, and combinations thereof.

In some embodiments, the biphasic topical composition contains ingredients effective as a bug repellant.

In some embodiments, the biphasic topical composition contains ingredients effective to treat itch.

In certain embodiments, the active ingredient to treat a fibrotic skin condition is part of Phase A and is a topical vitamin D analog. In certain embodiments, the active ingredient to treat a fibrotic skin condition is part of Phase B and is adenosine A2A receptor antagonist.

In some embodiments, the biphasic topical composition has a pH of less than about 7.5. In some embodiments, the biphasic topical composition has a pH of less than about 5.5. In some embodiments, the biphasic topical composition has a pH selected from the group consisting of about 4.0 to about 7.5, about 4.5 to about 7.5, and about 4.4 to about 7.5.

In some embodiments, the biphasic topical composition is part of an anti-aging regimen. In some embodiments, the biphasic topical composition is part of regimen for after sun care. In some embodiments, the biphasic topical composition is part of a photoprotective regimen. In some embodiments, the photoprotective regimen is a sunblock regimen or a sunscreen. In some embodiments, the biphasic topical composition is part of regimen for skin lightening. In some embodiments, the biphasic topical composition is part of regimen for skin brightening. In some embodiments, the biphasic topical composition is part of regimen for acne treatment. In some embodiments, the biphasic topical composition is part of regimen for inflammation treatment. In some embodiments, the biphasic topical composition is part of a color cosmetic regimen. In some embodiments, the biphasic topical composition is part of a hair treatment regimen. In some embodiments, the antioxidant composition is part of a scalp treatment regimen.

A wide variety of methods may be used for preparing the biphasic topical composition described herein. Broadly speaking, the compositions may be prepared by combining the components of the formulation, as described herein, at a temperature and for a time sufficient to provide a pharmaceutically acceptable composition.

### Methods of Using Compositions Described Herein

Embodiments described herein are directed to methods of treating uneven skin tone in a subject in need thereof comprising: shaking a biphasic topical composition comprising a Phase A including castor oil, one or more additional non-polar solvents, and one or more oil soluble active ingredients, and a Phase B including ethanol, one or more additional polar solvents, and one or more polar or water soluble active ingredients to form a mixed biphasic topical composition, and applying the mixed biphasic topical composition to the skin of the subject, wherein the ratio of castor oil to ethanol in the biphasic topical composition is about 1:1 to about 3:1.

In embodiments directed to methods of treating uneven skin tone in a subject in need thereof, the oil soluble active ingredient of Phase A is selected from the group consisting of phenethyl resorcinol, hydroquinone, kojic acid, deoxyarbutin and all derivatives thereof, sclareolide (under trade name SymBright), retinoids such as retinol, hydroxy pinacolone retinoate, retinal, mequinol, N-acetyl-4-S-Cysteaminylphenol, azelaic acid, cysteamine, mandelic acid, gentisic acid, flavonoids, aloesin, tetrahydrodiferuloylmethane, and combinations thereof.

In embodiments directed to methods of treating uneven skin tone in a subject in need thereof, the polar or water soluble active ingredient of Phase B is selected from the group consisting of active L-ascorbic acid, niacinamide, licorice root extracts, papaya extracts, ellagic acid, tranexamic acid, papain, arbutin, peptides (such as tetrapeptide-30), and combinations thereof.

Embodiments described herein are directed to methods of treating acne in a subject in need thereof comprising: shaking a biphasic topical composition comprising a Phase A including castor oil, one or more additional non-polar solvents, and one or more oil soluble active ingredients, and a Phase B including ethanol, one or more additional polar solvents, and one or more polar or water soluble active ingredients to form a mixed biphasic topical composition, and applying the mixed biphasic topical composition to the skin of the subject, wherein the ratio of castor oil to ethanol in the biphasic topical composition is about 1:1 to about 3:1.

In embodiments directed to methods of treating acne in a subject in need thereof, the oil soluble active ingredient of Phase A is selected from the group consisting of salicylic acid, retinoids such as retinol, retinal, hydroxy pinacolone retinoate, sulfur, tea tree oil, bakuchiol, bakutrol, honokiol, phloretin, and combinations thereof.

In embodiments directed to methods of treating acne in a subject in need thereof, the polar or water soluble-active ingredient of Phase B is selected from the group consisting of active L-ascorbic acid, caprylol glycine, undecylenoyl glycine, Phytosphingosine, peroxides, mandelic acid, and combinations thereof.

Embodiments described herein are directed to methods of lightening or brightening the skin in a subject in need thereof comprising: shaking a biphasic topical composition comprising a Phase A including castor oil, one or more additional non-polar solvents, and one or more oil soluble active ingredients, and a Phase B including ethanol, one or more additional polar solvents, and one or more polar or water soluble active ingredients to form a mixed biphasic topical composition, and applying the mixed biphasic topical composition to the skin of the subject, wherein the ratio of castor oil to ethanol in the biphasic topical composition is about 1:1 to about 3:1.

In embodiments directed to methods of lightening or brightening the skin in a subject in need thereof, the oil soluble active ingredient of Phase A is selected from the group consisting of urea derivatives, sclareolide, retinoids, and combinations thereof.

In embodiments directed to methods of lightening or brightening the skin in a subject in need thereof, the polar or water soluble active ingredient of Phase B is selected from the group consisting of azelaic acid, bearberry extract, arbutin, deoxyarbutin, Glycyrrhiza glabra (Licorice) root extract, kojic acid, peat extract, niacinamide, ascorbic acid, Centella asiatica extract, citric acid, benzoyl peroxide, carbamide peroxide, mandelic acid or other alpha-hydroxy acids, and their derivatives and combinations thereof.

Embodiments described herein are directed to methods of protecting a subject's skin from sun damage comprising: shaking a biphasic topical composition comprising a Phase A including castor oil, one or more additional non-polar solvents, and one or more oil soluble active ingredients, and a Phase B including ethanol, one or more additional polar solvents, and one or more polar or water soluble active ingredients to form a mixed biphasic topical composition, and applying the mixed biphasic topical composition to the skin of the subject, wherein the ratio of castor oil to ethanol in the biphasic topical composition is about 1:1 to about 3:1.

In embodiments directed to methods of protecting a subject's skin from sun damage, the oil soluble active ingredient of Phase A is selected from the group consisting of Homosalate, Ethylhexyl Salicylate, Octinozate, Octocrylene, Butyl Methoxydibenzoylmethane, ZnO, TiO2, bis-Ethylhexyloxyphenol Methoxyphenyl Triazine (Tinosorb® S), Diethylamino Hydroxybenzoyl Hexyl Benzoate (Uvinul® A Plus), Drometrizole Trisiloxane, Menthyl Anthranilate, Zinc Oxide (nano), lipophilic phase (Z-Cote®), Zinc Oxide (nano), lipophilic phase (Z-Cote® HP1), 4-Methylbenzylidene Camphor, Benzophenone-3, Benzophenone-4 (Uvinul® MS 40), Diethylhexyl Butamido Triazone, Ethylhexyl Methoxycinnamate (Uvinul® MC 80), Ethylhexyl Triazone (Uvinul® T 150), Ethylhexyl dimethyl PABA, Homomenthyl Salicylate, Isoamyl p-Methoxycinnamate, Octocrylene (Uvinul® N 539 T), PEG-25 PABA, Polysilicone-15, Titanium Dioxide (nano), lipophilic phase, Tris Biphenyl Triazine (nano), (Tinosorb® A2B), and combinations thereof.

In embodiments directed to methods of protecting a subject's skin from sun damage, the polar or water soluble active ingredient of Phase B is selected from the group consisting of Bis-Ethylhexyloxyphenol Methoxyphenyl Triazine, (Tinosorb® S Lite Aqua), Methylene Bis-Benzotriazolyl Tetramethylbutylphenol (nano) (Tinosorb® M), Phenylbenzimidazol Sulfonic Acid, Terephthalylidene Dicamphor Sulfonic Acid, Disodium Phenyl Dibenzimidazole Tetrasulfonate, Titanium Dioxide (nano), hydrophilic phase, Zinc Oxide (nano), hydrophilic phase, and combinations thereof.

Embodiments described herein are directed to methods of protecting a subject's skin from the effects of aging comprising: shaking a biphasic topical composition comprising a Phase A including castor oil, one or more additional non-polar solvents, and one or more oil soluble active ingredients, and a Phase B including ethanol, one or more additional polar solvents, and one or more polar or water soluble active ingredients to form a mixed biphasic topical composition, and applying the mixed biphasic topical composition to the skin of the subject, wherein the ratio of castor oil to ethanol in the biphasic topical composition is about 1:1 to about 3:1.

In embodiments directed to methods of protecting a subject's skin from the effects of aging, the oil soluble active ingredient of Phase A is selected from the group consisting of retinoids, oil-soluble vitamins and their derivatives, multifunctional oils, e.g. Karanja oil, Karanja ester (karanjate), Almond oil, CBD Oil, Coconut Oil, Sunflower Oil, Safflower oil, Hemp Oil, Jojoba Oil, Macadamia Nut Oil, and combinations thereof.

In embodiments directed to methods of protecting a subject's skin from the effects of aging, the polar or water soluble active ingredient of Phase B is selected from the group consisting of niacinamide, vitamin C, botanical plant extracts, amino acids, peptides, plant growth factors, urea derivatives, and combinations thereof.

Embodiments described herein are directed to methods of improving the look of the skin comprising: shaking a biphasic topical composition comprising a Phase A including castor oil, one or more additional non-polar solvents, and one or more oil soluble active ingredients, and a Phase B including ethanol, one or more additional polar solvents, and one or more polar or water soluble active ingredients to form a mixed biphasic topical composition, and applying the mixed biphasic topical composition to the skin of the subject, wherein the ratio of castor oil to ethanol in the biphasic topical composition is about 1:1 to about 3:1.

In embodiments directed to methods of improving the look of skin, the oil soluble active ingredient of Phase A is selected from the group consisting of karanja oil, karanja ester (Karanjate), almond oil, CBD Oil, Coconut Oil, Sunflower Oil, Safflower oil, Hemp Oil, Jojoba Oil, Macadamia Nut Oil, coated Iron Oxides, Titanium dioxide compatible with oil phase, and combinations thereof.

In embodiments directed to methods of improving the look of skin, the polar or water soluble active ingredient of Phase B is selected from the group consisting of niacinamide, peptides, amino acids, and combinations thereof.

In embodiments described herein, improving the look of skin is an improvement in a characteristic of the skin. In some embodiments, the characteristic of the skin is selected from the group consisting of firmness, elasticity, fine lines, wrinkles, skin texture, skin tone, appearance, and any combination thereof. In some embodiments, improving the look of the skin results in smoother, firmer, young-looking skin. In some embodiments, improving the look of the skin results in a brighter complexion, improved texture, and even-looking skin.

Embodiments described herein are directed to methods of improving the appearance of the skin comprising: shaking a biphasic topical composition comprising a Phase A including castor oil, one or more additional non-polar solvents, and one or more oil soluble active ingredients, and a Phase B including ethanol, one or more additional polar solvents, and one or more polar or water soluble active ingredients to form a mixed biphasic topical composition, and applying the mixed biphasic topical composition to the skin of the subject, wherein the ratio of castor oil to ethanol in the biphasic topical composition is about 1:1 to about 3:1.

In embodiments directed to methods of improving the appearance of skin, the oil soluble active ingredient of Phase A is selected from the group consisting of ascorbic acid derivatives, oil soluble vitamins, urea derivatives, and combinations thereof.

In embodiments directed to methods of improving the appearance of skin, the polar or water soluble active ingredient of Phase B is selected from the group consisting of vitamin B3 compounds, niacinamide, azelaic acid, butyl hydroxyanisole, gallic acid and its derivatives, glycyrrhizinic acid salts, hydroquinone, kojic acid, arbutin, deoxyarbutin, ferulic acid, ferulic acid dimer, mulberry extract, Ovaliss ((S)-5,6,6a,7-Tetrahydro-1,2,9,10-tetramethoxy-6-methyl-4H-dibenzo[de,g] quinoline, D-Glucopyranose, oligomeric, C10-16-alkyl glycosides, water, ethyl alcohol, and glycerin, Whey protein, MPC (Milk protein complex), Sesaflash (Glycerin, Acrylates copolymer, PVP/polycarbamyl polyglycol ester, Hydrolyzed Sesame Protein PG—propyl methylsilanediol), Majestem (Glycerin, Leontopodium Alpinum Callus Culture Extract and xanthan gum), Idealift (butylene glycol, sorbitan laurate, hydroxyethylcellulose, acetyl dipeptide-1 cetyl ester), and combinations thereof.

In embodiments described herein, improving the appearance of the skin results in smoother skin, firmer skin, softer skin, brighter complexion, improved texture of the skin, even-looking skin, improvement of discoloration, disappearance of blemishes, decreased redness, or younger-looking skin. In some embodiments, improving the appearance of the skin results in an anti-inflammatory effect. In some embodiments, improving the appearance of the skin results in an increase in elasticity of the skin, a decrease in the fine lines of the skin, a decrease in the wrinkles of the skin, a more consistent skin tone, and any combination thereof.

Embodiments described herein are directed to methods of treating fibrotic skin conditions in a subject in need thereof, comprising: shaking a biphasic topical composition comprising a Phase A including castor oil, one or more additional non-polar solvents, and one or more oil soluble active ingredients, and a Phase B including ethanol, one or more additional polar solvents, and one or more polar or water soluble active ingredients to form a mixed biphasic topical composition, and applying the mixed biphasic topical composition to the skin of the subject, wherein the ratio of castor oil to ethanol in the biphasic topical composition is about 1:1 to about 3:1.

In embodiments directed to methods of treating fibrotic skin conditions in a subject in need thereof, the oil soluble active ingredient of Phase A is a topical vitamin D analog.

In embodiments directed to methods of treating fibrotic skin conditions in a subject in need thereof, the polar or water soluble active ingredient of Phase B is adenosine A2A receptor antagonist.

In embodiments described herein, the fibrotic skin condition is selected from the group consisting of scleroderma, nephrogenic fibrosing dermopathy, mixed connective tissue disease, scleromyxedema, scleroderma, eosinophilic fasciitis, and combinations thereof.

Embodiments described herein are directed to methods of treating the hair, comprising: shaking a biphasic topical composition comprising a Phase A including castor oil, one or more additional non-polar solvents, and one or more oil soluble active ingredients, and a Phase B including ethanol, one or more additional polar solvents, and one or more polar or water soluble active ingredients to form a mixed biphasic topical composition, and applying the mixed biphasic topical composition to the skin of the subject, wherein the ratio of castor oil to ethanol in the biphasic topical composition is about 1:1 to about 3:1.

In embodiments directed to methods of treating the hair, the oil soluble active ingredient of Phase A is selected from the group consisting of lanolin, silicone, dimethicone, ceramide, fatty acids, natural plant and animal oils, mineral oil, olive oil, argan oil, oil-soluble vitamins, and combinations thereof.

In embodiments directed to methods of treating the hair, the polar or water soluble active ingredient of Phase B is selected from the group consisting of proteins, amino acids, collagen, and keratin, vitamins, betaine surfactants, amine oxide surfactants, eggs, milk, polyquaternium, In some embodiments, the protein is selected from the group consisting of collagen, keratin, soy protein, wheat protein, bean palmitate, ascorbic acid polypeptide, the amino acids, casein, cholecalciferol polypeptide, rice protein, silk protein, gluten protein, lysine, acetyl glucosamine, actin, actizyme, albumen, conchiorin protein, corn protein, egg protein, elastin, fibronectin, gadidae protein, hemoglobin, hexapeptide-21, lactalalbumin, lupine protein, maple sycamore protein, milk protein, myristoyl pentapeptide-8, myristoyl tetrapeptide-8, oat protein, oligopeptide 10, palmitoyl hexapeptide-14, palmitoyl oligopeptide, palmitoyl tetrapeptide-7, pea protein, potato protein, reticulin, rice bran protein, serum protein, sweet almond protein, tetrapeptide-16, vegetable protein, yeast protein, palmitoyl oligopeptide, pantothenic acid polypeptides, milk solids, sericin, albumen, amylase, amyloglucosidase, arginine, bromelain, catalase, gelatin, zein, crystallins, cytochrome C, deoxyribonuclease, gliadin, glucose oxidase, glycoproteins, lactoferrin, lactoglubulin, lactoperoxidase, lipase, nisin, oxido reductases, papain, pepsin, subtilisin, sutilains, and their derivatives and combinations thereof.

In embodiments described herein, the treatment of the hair results in improvement in a characteristic of the hair. In embodiments described herein, the characteristic of the hair is selected from the group consisting of shine, texture, fullness, smoothness, density, and combinations thereof.

Embodiments described herein are directed to methods of improving the appearance of the hair, comprising: shaking a biphasic topical composition comprising a Phase A including castor oil, one or more additional non-polar solvents, and one or more oil soluble active ingredients, and a Phase B including ethanol, one or more additional polar solvents, and one or more polar or water soluble active ingredients to form a mixed biphasic topical composition, and applying the mixed biphasic topical composition to the skin of the subject, wherein the ratio of castor oil to ethanol in the biphasic topical composition is about 1:1 to about 3:1.

In embodiments directed to methods of improving the appearance of the hair, the oil soluble active ingredient of Phase A is selected from the group consisting of argan oil, olive oil, karanja oil, karanja ester (karanjate), ceramides, and combinations thereof.

In embodiments directed to methods of improving the appearance of the hair, the polar or water soluble active ingredient of Phase B is selected from the group consisting of panthenol, amino acids, plant extracts, peptides, and combinations thereof.

In embodiments described herein, improving the appearance of the hair results in smoother hair, softer hair, brighter hair, improved texture of the hair, shiner hair, fuller hair, or more vibrant hair.

In embodiments described herein, the subject is an infant, a child, an adolescent, or an adult.

In some embodiments, the biphasic topical composition can be applied to the skin one, two, three, four, five or more times each day, and application can be carried out for a period of at least 1 month, 2 months, 3 months, 4 months, 6 months, 8 months or 12 months.

In some embodiments, the biphasic topical composition may be administered once, as needed, once daily, twice daily, three times a day, once a week, twice a week, every other week, every other day, or the like for one or more dosing cycles. A dosing cycle may comprise administration for about 1 week, about 2 weeks, about 3 weeks, about 4 weeks, about 5 weeks, about 6 weeks, about 7 weeks, about 8 weeks, about 9 weeks, or about 10 weeks. After this cycle, a subsequent cycle may begin approximately 1, 2, 3, 4, 5, 6, 7, 8, 9, 10, 11, or 12 weeks later. The treatment regime may comprise 1, 2, 3, 4, 5, or 6 cycles, each cycle being spaced apart by approximately 1, 2, 3, 4, 5, 6, 7, 8, 9, 10, 11, or 12 weeks.

In some embodiments, the methods may comprise a variety of additional steps including, for example, cleaning the surface tissue at the site of applying, abrading, microdermabrasion, toning, and the like.

### Methods of Preparing Compositions Described Herein

Broadly speaking, the compositions may be prepared by combining together the components of the formulation, as described herein, at a temperature and for a time sufficient to provide a pharmaceutically acceptable composition.

In embodiments described herein, the process for preparation of polyol and oil biphasic topical composition includes: mixing pentylene glycol and glycerin together to create Phase B, heating to 80-85° C., adding L-Ascorbic Acid or Niacinamide to the Phase B mixture, mixing with a stir bar until dissolved, cooling Phase B to room temperature, adding any additional polyol soluble ingredients that do not need heating and then adding ethanol with mixing. Phase A is created by mixing oils and oil soluble ingredients together. To create the biphasic topical composition both Phase A and Phase B are combined in the necessary ratio.

In embodiments described herein, the process for preparation of water and oil biphasic systems topical composition includes: mixing oil phase ingredients to create Phase A, separately combining ingredients in water phase to create Phase B, mixing until all items are dissolved, adding fragrance to Phase B. To create the biphasic topical composition both Phase A and Phase B are combined in the necessary ratio.

The subject matter is now described with reference to the following examples. These examples are provided for the purpose of illustration only and the claims should in no way be construed as being limited to these examples, but rather should be construed to encompass any and all variations which become evident as a result of the teaching provided herein. Those of skill in the art will readily recognize a variety of non-critical parameters that could be changed or modified to yield essentially similar results.

### EXAMPLES

### Example 1: Formulations

By attenuating the percent of oil phase and the ratio of castor oil to ethanol the time to separate the sample after mixing was optimized. The bi-phasic combinations have niacinamide in Phase B and with or without a hydrophobically modified ascorbic acid in Phase A. Both ingredients are known to even skin tone. Formulations tested are provided in Table 1.

TABLE 1
Example composition Ex. 1-7.

| | | | Example | | | | | | |
|---|---|---|---|---|---|---|---|---|---|
| | | | 1 | 2 | 3 | 4 | 5 | 6 | 7 |
| | role | INCI | % | % | % | % | % | % | % |
| Phase | non-polar | Isohexadecane | 16.67 | 14.04 | 12 | 14.84 | 20.54 | 10.54 | 22.54 |
| A | solvents | Ethyl linoleate | 16.67 | 5.46 | 5.46 | 5.46 | 5.46 | 5.46 | 5.46 |
| | | C12-15 Alkyl benzoate | 16.66 | 11.0 | 11.0 | 11.0 | 11.0 | 11.0 | 11.0 |
| | Oil soluble | Tetrahexyldecyl | | 10.0 | | | | 10.0 | |
| | ingredients | Ascorbate | | | | | | | |
| | oil with hydrophilic | Castor Oil | | 2.5 | 12.7 | 12.7 | 6.0 | 6.0 | 6.0 |
| | functionalization | | | | | | | | |
| Phase | water miscible polar | Ethanol | | 5.0 | 8.74 | 5.0 | 5.0 | 5.0 | 5.0 |
| B | organic solvent | | | | | | | | |
| | Polar Phase | Glycerin | 40.0 | 40.0 | 40.0 | 40.0 | 40.0 | 40.0 | 35.5 |
| | | Pentylene Glycol | 7.5 | 7.5 | 7.5 | 7.5 | 7.5 | 7.5 | 7.5 |
| | Polyol soluble | Niacinamide | 2.5 | 2.5 | 2.5 | 2.5 | 2.5 | 2.5 | 5.0 |
| | ingredients | | | | | | | | |
| | Fragrance | essential oil | | 2.0 | 0.1 | 1.0 | 2.0 | 2.0 | 2.0 |
| | | solution in | | | | | | | |
| | | pentylene glycol | | | | | | | |
| | | total | 100 | 100 | 100 | 100 | 100 | 100 | 100 |

Table 2 contains additional formulations with active L-ascorbic acid in Phase B and other ingredients that even skin tone in Phase A.

TABLE 2
Example compositions 8-11.

| | | | Example | | | |
|---|---|---|---|---|---|---|
| | | | 8 | 9 | 10 | 11 |
| | role | INCI | % | % | % | % |
| Phase | non-polar | Isohexadecane | 25 | 22.5 | 14.63 | 17.5 |
| A | solvents | Ethyl linoleate | | | 5.46 | 5.46 |
| | | C12-15 Alkyl benzoate | 25.0 | 25.0 | 5.54 | 5.54 |
| | | Caprylic/Capric | | | 2.27 | |
| | | triglycerides | | | | |
| | | sesquiterpene | | | 0.6 | |
| | | derivative and/or urea | | | | |
| | | or its derivatives | | | | |
| | | Tocopherol acetate | | | 1.0 | 1.0 |
| | oil with hydrophilic | Castor Oil | | 2.5 | 6.0 | 6.0 |
| | functionalization | | | | | |
| Phase | water miscible polar | Ethanol | | 5.0 | 5.0 | 5.0 |
| B | organic solvent | | | | | |
| | Polar Phase | Glycerin | 40.0 | 30.0 | 40.0 | 47.5 |
| | | Water | | | | |
| | | Pentylene Glycol | 5.0 | 10.0 | 7.5 | 7.5 |
| | | L-ascorbic acid | 5.0 | 5.0 | 10.0 | 2.5 |
| | Fragrance | essential oil solution | | | 2.0 | 2.0 |
| | | in pentylene glycol | | | | |
| | | total | 100 | 100 | 100 | 100 |

Table 3 contains additional formulations with active L-ascorbic acid in Phase B and other ingredients that even skin tone in Phase A.

TABLE 3
Inventive compositions 12-15

| | | | Example | | | |
|---|---|---|---|---|---|---|
| | | | 12 | 13 | 14 | 15 |
| | role | INCI | % | % | % | % |
| Phase | non-polar | Isohexadecane | 22.5 | 14.63 | 14.0 | 25.0 |
| A | solvents | Ethyl linoleate | | 5.46 | 5.5 | |
| | | C12-15 Alkyl benzoate | 25.0 | 5.54 | 11.0 | 25.0 |
| | | Caprylic/Capric | | 2.27 | | |
| | | triglycerides | | | | |
| | | sesquiterpene | | 0.6 | | |
| | | derivative and/or urea | | | | |
| | | or its derivatives | | | | |
| | | Tocopherol acetate | | 1.0 | | |
| | oil with hydrophilic | Castor Oil | 2.5 | 6.0 | 2.5 | |
| | functionalization | | | | | |
| Phase | water miscible | Ethanol | 5.0 | 5.0 | 5.0 | |
| B | polar organic solvent | | | | | |
| | | Water | 40.0 | 47.5 | 52.0 | 45.0 |
| | | L-ascorbic acid | 5.0 | 10.0 | 10.0 | 5.0 |
| | Fragrance | essential oil solution | | 2.0 | | |
| | | in pentylene glycol | | | | |
| | | total | 100 | 100 | 100 | 100 |

### Example 2: Phase Separation

Examples were tested for the time until visible separation occurred between Phase A and Phase B after mixing. Method A used for testing time to separation includes:

1. Add 25 uL of Amber orange (Biolie www.biolie.com) to Phase A as an indicator to clearly visualize separation of phases.

2. Set the Vortex-2 Genie to speed 10, with the continuous ON button.

3. Weigh 20 g total volume of formula in a scintillation vial.

4. Place the bi-phase solutions on the vortex for 10 seconds.

5. Take off the solutions from the vortex, and record time of visual separation.

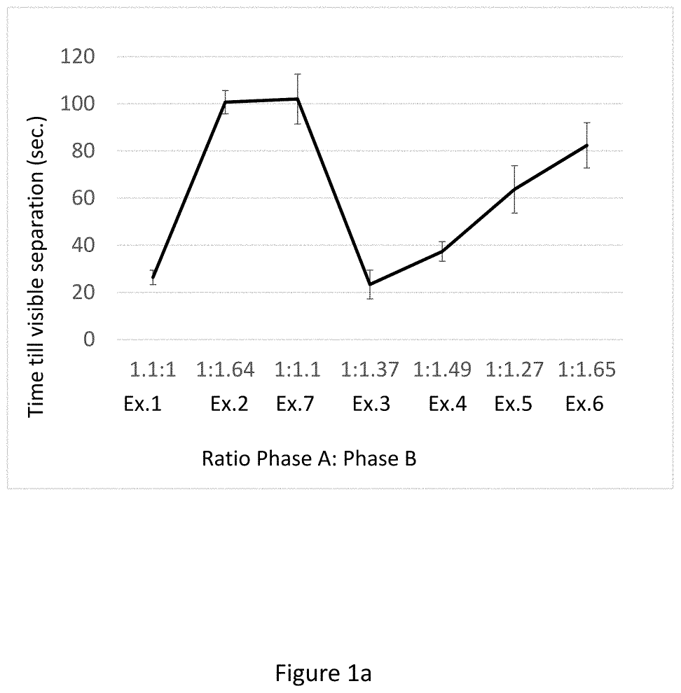

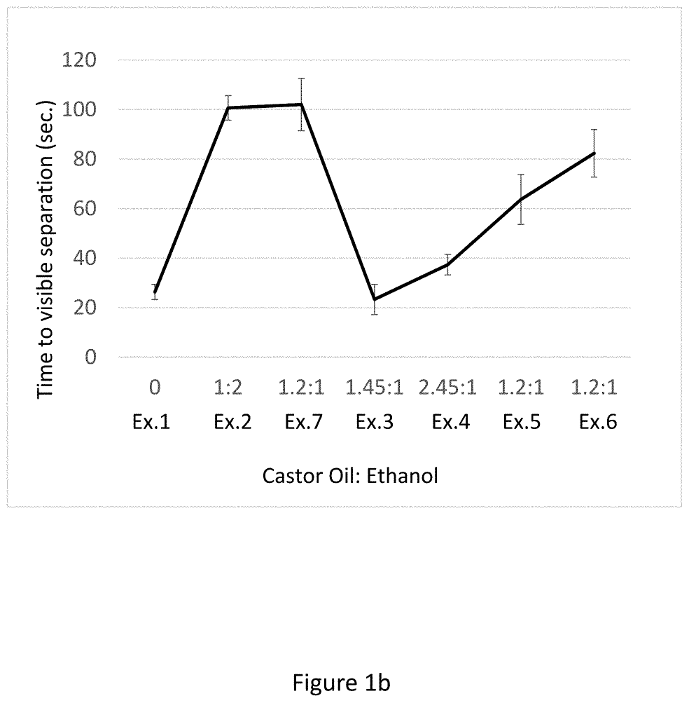

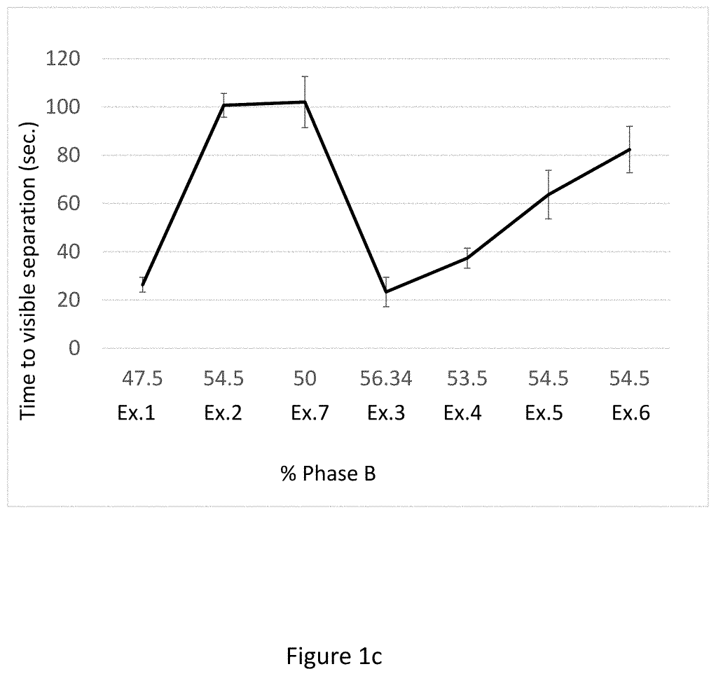

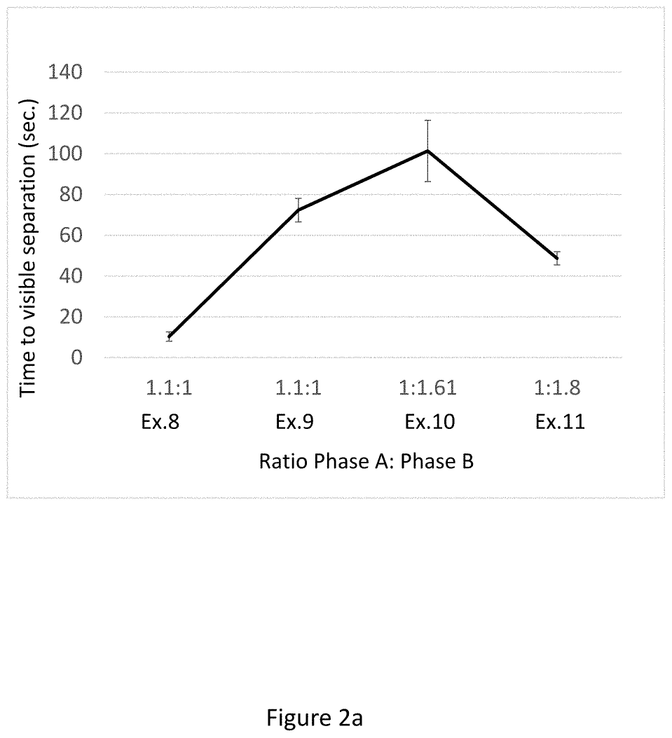

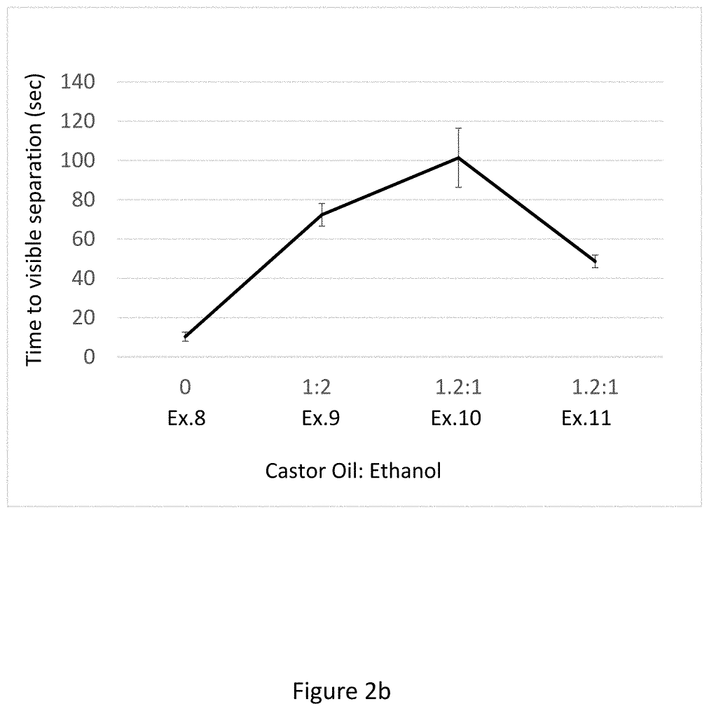

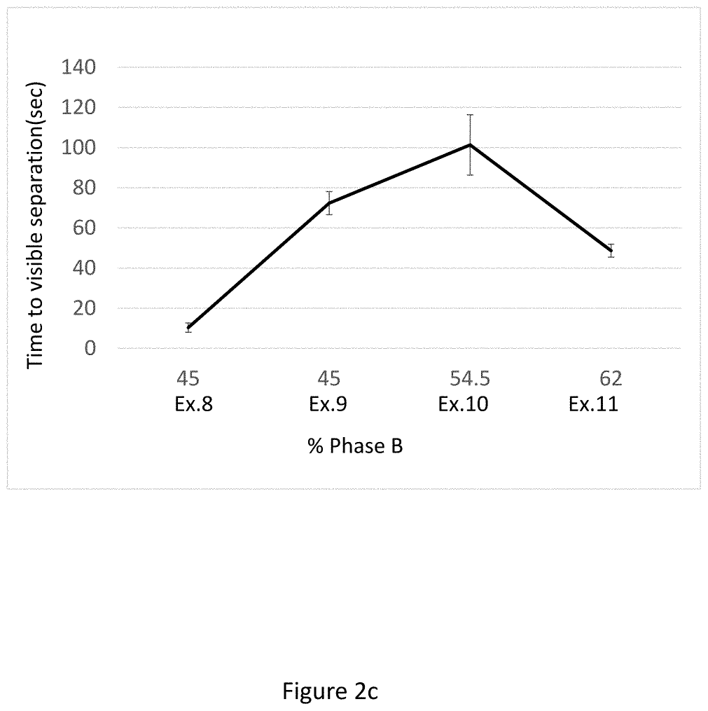

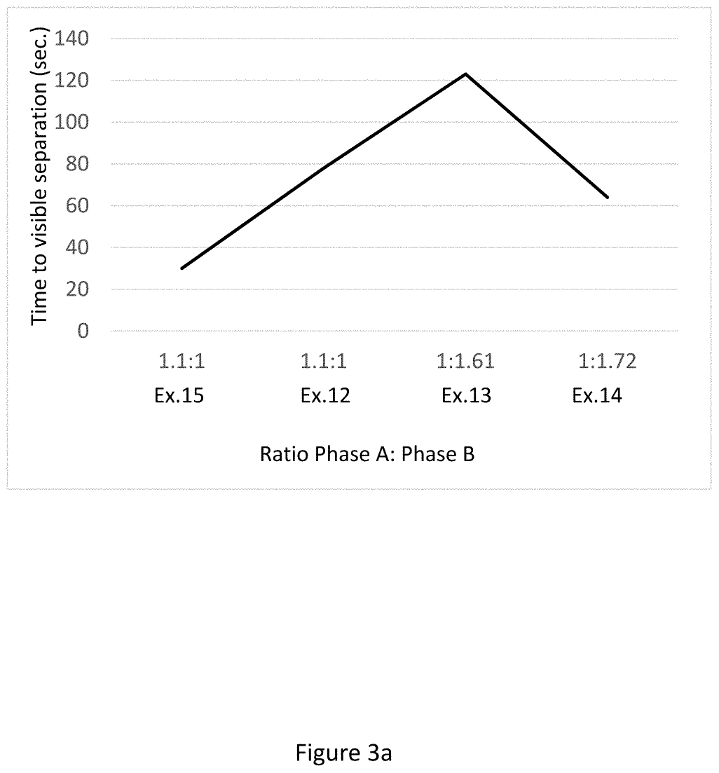

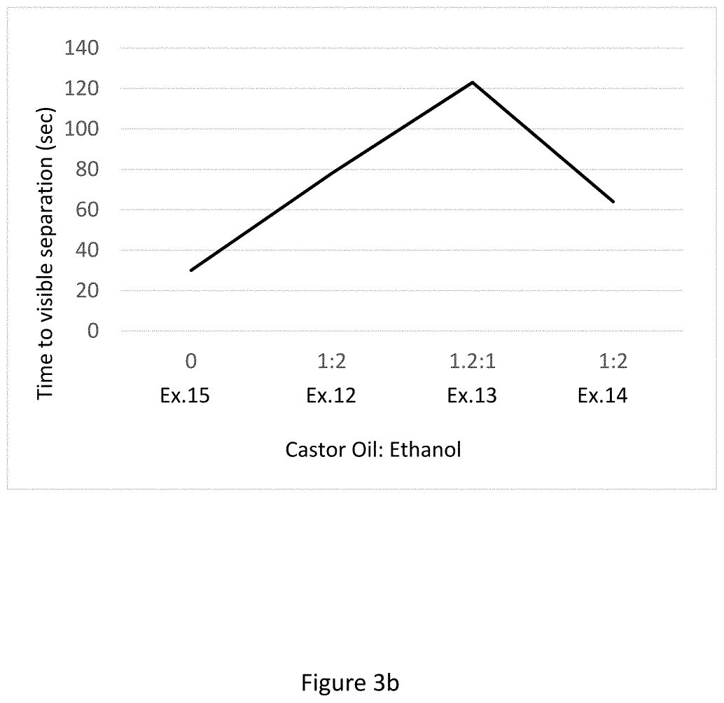

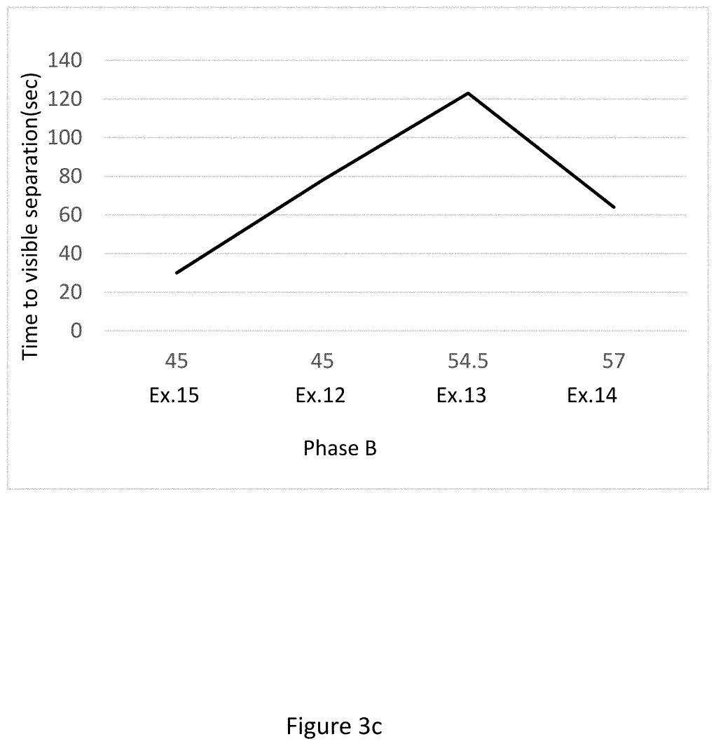

Results for Examples 1-7 are provided in Table 4 and FIGS. 1a-1c. Results for Examples 8-11 are provided in Table 5 and FIGS. 2a-2b. Results for Examples 12-15 are provided in Table 7 and FIGS. 3a-2b.

TABLE 4
Time for visible separation after mixing in Ex. 1-7

| | % | % | Ratio | Ratio | | | | | |
|---|---|---|---|---|---|---|---|---|---|
| | Phase | Phase | Castor | Phase | Trial 1 | Trial 2 | Trial 3 | | |
| Ex. | A | B | Oil:ethanol | A:Phase B | (sec.) | (sec.) | (sec.) | Ave. | STD |
| 1 | 50.01 | 47.5 | 0 | 1.1:1 | 29 | 27 | 23 | 26.3 | 3.1 |
| 2 | 33 | 54.5 | 1:2 | 1:1.64 | 95 | 104 | 103 | 100.7 | 4.9 |
| 7 | 45 | 50 | 1.2:1 | 1:1.1 | 98 | 114 | 94 | 102 | 10.6 |
| 3 | 41.16 | 56.34 | 1.45:1 | 1:1.37 | 18 | 22 | 30 | 23.3 | 6.1 |
| 4 | 36 | 53.5 | 2.45:1 | 1:1.49 | 36 | 42 | 34 | 37.3 | 4.2 |
| 5 | 43 | 54.5 | 1.2:1 | 1:1.27 | 73 | 65 | 53 | 63.7 | 10.1 |
| 6 | 33 | 54.5 | 1.2:1 | 1:1.65 | 72 | 84 | 91 | 82.3 | 9.6 |

For examples with niacinamide in Phase B the addition of castor oil and ethanol increased the time to visible separation, as determined by Method A. FIGS. 1a-1c graphically present the time to separation compared to ratio of Phase A: Phase B (FIG. 1a), ratio of castor oil to ethanol (FIG. 1b), and % Phase B (FIG. 1c). In general, for bi-phasic compositions with a higher percentage of Phase B separation times are increased maximally when the ratio of castor oil to ethanol is 1:2 favoring more ethanol (i.e. Ex. 2). However, acceptable separation times are also achieved with a ratio of 1.2:1 castor oil to ethanol (see Ex. 6 and 7). In contrast separation times were faster (or reduced) for samples with high percentage of Phase B and with higher ratios of castor oil to ethanol (from 1.45:1 to 2.45:1, see Ex 4, 5) and were comparable to formulations without the castor oil and ethanol blend (Ex. 1).

TABLE 5
Time for visible separation after mixing in Ex. 8-11

| | % | % | | Ratio | | | | | |
|---|---|---|---|---|---|---|---|---|---|
| | Phase | Phase | Ratio | Phase | Trial 1 | Trial 2 | Trial 3 | | |
| Ex. | A | B | Castor:ethanol | A:Phase B | (sec) | (sec) | (sec) | Ave. | STD |
| 8 | 50 | 45 | 0 | 1.1:1 | 9 | 9 | 13 | 10.3 | 2.3 |
| 9 | 50 | 45 | 1:2 | 1.1:1 | 69 | 69 | 79 | 72.3 | 5.8 |
| 10 | 33.9 | 54.5 | 1.2:1 | 1:1.61 | 100 | 117 | 87 | 101.3 | 15.0 |
| 11 | 34.5 | 62 | 1.2:1 | 1:1.8 | 50 | 51 | 45 | 48.7 | 3.2 |

For formulations with L-ascorbic acid in Phase B complications arose to the fact that the presence of ethanol in Phase B caused the L-ascorbic acid to precipitate out of solution over time (see Table 6). Interestingly, the upper limit for combinations with L-ascorbic acid with castor oil and ethanol is less than 5% L-ascorbic acid. FIGS. 2a-1c graphically present the time to separation of ascorbic acid containing biphasic Examples 8-11 as a function of the ratio of Phase A: Phase B (FIG. 2a), ratio of castor oil to ethanol (FIG. 2b), and % Phase B (FIG. 2c). In terms of observed separation time the addition of castor oil and ethanol increased the separation time of the inventive compositions from 10 seconds (Ex. 8) to >40 sec (Ex. 9-Ex. 11), longer separation times were observed when the ratio of castor oil to ethanol favored the ethanol (1:1.1 to 1:2) for formulations with more Phase B and for formulations with higher ratio of castor oil to ethanol (1.2:1, Ex. 10).

Formulations with ethanol and ascorbic acid showed a formation of a precipitate caused by the addition of ethanol into Phase B. This precipitate could be offset by lowering the percent of L-ascorbic acid used in the formulation to less than 5% (see Table 6). Precipitation of niacinamide was not observed in polyol, ethanol and oil formulations. Additionally, no ascorbic acid precipitated in water in oil systems with or without ethanol.

TABLE 6
Stability observations at room temperature.

| Ex. | 1 week | 2 week | 3 week | 4 week |
|---|---|---|---|---|
| 8 | slightly hazy | slightly hazy | slightly hazy | slightly hazy |
| 9 | clear | clear | clear | clear |
| 1 | slightly hazy | slightly hazy | slightly hazy | slightly hazy |
| 2 | clear | clear | clear | clear |
| 7 | slightly hazy | slightly hazy | slightly hazy | slightly hazy |
| 10 | precipitation | precipitation | precipitation | precipitation |
| 14 | slightly hazy | slightly hazy | slightly hazy | slightly hazy |
| 3 | clear | clear | clear | clear |
| 4 | clear | clear | clear | clear |
| 5 | slightly hazy | slightly hazy | slightly hazy | slightly hazy |
| 6 | clear | clear | clear | clear |

Note:
if precipitate was observed onset is recorded below. If no precipitate was observed that is recorded below. If solution is hazy, but there is no precipitate observed that is recorded as hazy.

FIGS. 3a-1c graphically present the time to separation of ascorbic acid containing biphasic Examples 12-15 as a function of the ratio of Phase A: Phase B (FIG. 3a), ratio of castor oil to ethanol (FIG. 3b), and % Phase B (FIG. 3c). In water and oil systems the addition of castor oil and ethanol increased the separation time, without castor oil and ethanol the separation time was rather fast at about 30 seconds (see Ex. 15 in Table 7 and FIGS. 3a-3c). In contrast to polyol and oil systems described above the longest separation times were recorded for combinations with higher ratio of castor oil to ethanol (1.2:1) and when higher percentage of water was present in the formulation (see Ex. 13 in Table 7 and FIGS. 3a-3c). Intermediate separation times were observed for castor oil to ethanol ratios of 1:2 when either a higher percentage of oil was present in the formulation (see Ex. 12 in Table 7 and FIGS. 3a-3c) or a higher percentage of water was present in the formulation (see Ex. 14 in Table 7 and FIGS. 3a-3c). Therefore, the optimal ratio of castor oil to ethanol for water and oil systems is 1:2 to 2:1.

TABLE 7
Time for visible separation after mixing in Ex. 12-15

| | Time to | | | Ratio of | Ratio of |
|---|---|---|---|---|---|
| | Separate | % Phase | % Phase | Castor | organic |
| Ex. | (seconds) | A | B | Oil:Ethanol | phase:water |
| 15 | 30 | 50 | 45 | 0 | 1.1:1 |
| 12 | 78 | 50 | 45 | 1:2 | 1.1:1 |
| 13 | 123 | 33.92 | 54.5 | 1.2:1 | 1:1.61 |
| 14 | 64 | 33 | 57 | 1:2 | 1:1.72 |

Note:
only a single reading was taken as full separation took more than 24 hours.

### Example 3: Process for Preparation of Polyol and Oil Bi-Phasic Systems

The process used to prepare the polyol and oil biphasic composition includes: 1. Mix pentylene glycol and glycerin together heat to 80-85° C.; 2. Add L-Ascorbic Acid or Niacinamide to Phase B mixture and mix with a stir bar until dissolved; 3. Cool Phase B to room temperature, add any additional polyol soluble ingredients that do not need heating and then add ethanol with mixing; 4. In a separate beaker mix oils and oil soluble ingredients together; and 5. When all phases complete combine phases in correct ratio for each example.

### Example 4: Process for Preparation of Water and Oil Bi-Phasic Systems

The process used to prepare the water and oil biphasic composition includes: 1. Combine oil phase ingredients in main vessel; 2. In a separate vessel combine ingredients in water phase and mix until all items are dissolved; 3. Add fragrance phase to water phase; and 4. Combine water and oil phase together.

### Example 5: Biphasic Sunless Tanning Formulations

Table 8 provides the Biphasic sunless tanning formulations according to examples 16-19.

TABLE 8
Example compositions 16-19.

| | | | Example | | | |
|---|---|---|---|---|---|---|
| | | | 16 | 17 | 18 | 19 |
| | Function | INCI | % | % | % | % |
| Phase | non-polar | Isohexadecane | 22.5 | 20.0 | 12.5 | 20.0 |
| A | solvents | Ethyl linoleate | | 5.5 | 5.5 | |
| | | C12-15 Alkyl benzoate | 20.0 | 15.0 | 10.0 | 20.0 |
| | | Caprylic/Capric | 5.0 | 7.5 | 2.5 | |
| | | triglycerides | | | | |
| | | Tocopherol acetate | 0.5 | 1.0 | 0.5 | 1.0 |
| | oil with hydrophilic | Castor Oil | 2.5 | 6.0 | 2.5 | |
| | functionalization | | | | | |
| Phase | water miscible polar | Ethanol | 5.0 | 5.0 | 5.0 | |
| B | organic solvent | | | | | |
| | | Water | 35.0 | 46.0 | 44.5 | 44.5 |
| | | DHA | 2.5 | 5.0 | 10.0 | 7.50 |
| | Polar Solvent | Pentylene Glycol | 5.0 | 5.0 | 5.0 | 5.0 |
| | pH adjustor | Citric Acid | 1.0 | 1.0 | 1.0 | 1.0 |
| | Fragrance | Fragrance | 1.0 | 1.0 | 1.0 | 1.0 |
| | | Total | 100 | 100 | 100 | 100 |

The disclosures of each and every patent, patent application, publication, and accession number cited herein are hereby incorporated herein by reference in their entirety.

While present disclosure has been disclosed with reference to various embodiments, it is apparent that other embodiments and variations of these may be devised by others skilled in the art without departing from the true spirit and scope of the disclosure. The appended claims are intended to be construed to include all such embodiments and equivalent variations.

1. A biphasic topical composition comprising:
a Phase A including castor oil and one or more additional non-polar solvents, and
a Phase B including ethanol and one or more additional polar solvents,
wherein the ratio of castor oil to ethanol is about 1:1 to about 3:1; and wherein the biphasic topical composition is free of surfactants and emulsifiers.

2. The biphasic topical composition of claim 1, wherein time of separation of Phase A and Phase B after mixing is between about 10 seconds to about 5 minutes.

3. The biphasic topical composition of claim 1, wherein the ratio of Phase A to Phase B is about 1:1.1 to about 1:1.65.

4. The biphasic topical composition of claim 1, wherein the ratio of castor oil to ethanol is about 1.2:1.

5. The biphasic topical composition of claim 1, wherein the percentage of Phase B in the biphasic topical composition is about 50% to about 65%.

6. The biphasic topical composition of claim 1, wherein the one or more additional non-polar solvents is selected from the group consisting of isohexadecane, ethyl linoleate, C12-15 alkyl benzoate, caprylic/capric triglycerides, and combinations thereof.

7. The biphasic topical composition of claim 1, wherein the one or more additional polar solvents is selected from the group consisting of glycerin, butylene glycol, pentylene glycol, polyol, water, and combinations thereof.

8. The biphasic topical composition of claim 1, wherein the one or more additional polar solvents is glycerin in an amount of about 10% to about 40%.

9. The biphasic topical composition of claim 1, wherein Phase B is substantially free of water.

10. The biphasic topical composition of claim 1, wherein the Phase A is in the amount of about 14% to about 54% of the biphasic topical composition.

11. The biphasic topical composition of claim 1, wherein the Phase B is in the amount of about 10% to about 50% of the biphasic topical composition.

12. The biphasic topical composition of claim 1, wherein Phase A further comprises oil soluble ingredients.

13. The biphasic topical composition of claim 12, wherein the oil soluble ingredients are in an amount of about 0.001% to about 20% of the biphasic topical composition.

14. The biphasic topical composition of claim 12, wherein the oil soluble ingredients are selected from the group consisting of hydrophobically modified ascorbic acid, salicylic acid, phenethyl resorcinol, vitamin E acetate, and combinations thereof.

15. The biphasic topical composition of claim 1, wherein Phase B further comprises water or polyol soluble ingredients.

16. The biphasic topical composition of claim 15, wherein the water or polyol soluble ingredients are in an amount of about 2.5% to about 20% of the biphasic topical composition.

17. The biphasic topical composition of claim 15, wherein the water or polyol soluble ingredients are selected from the group consisting of niacinamide, ascorbic acid, extracts, kojic acid, citric acid, benzoyl peroxide, carbamide peroxide, water/polyol soluble pharmaceuticals, and combinations thereof.

18. A method of treating uneven skin tone in a subject in need thereof comprising:
shaking a biphasic topical composition comprising a Phase A including castor oil, one or more additional non-polar solvents, and one or more oil soluble active ingredients, and a Phase B including ethanol, one or more additional polar solvents, and one or more polar or water soluble active ingredients to form a mixed biphasic topical composition, and
applying the mixed biphasic topical composition to the skin of the subject, wherein the ratio of castor oil to ethanol in the biphasic topical composition is about 1:1 to about 3.1.

19. A method of treating acne in a subject in need thereof comprising:
shaking a biphasic topical composition comprising a Phase A including castor oil, one or more additional non-polar solvents, and one or more oil soluble active ingredients, and a Phase B including ethanol, one or more additional polar solvents, and one or more polar or water soluble active ingredients to form a mixed biphasic topical composition, and
applying the mixed biphasic topical composition to the skin of the subject, wherein the ratio of castor oil to ethanol in the biphasic topical composition is about 1:1 to about 3.1.
